(19) United States
(12) Patent Application Publication
(10) Pub. No.: US 2002/0015707 A1
(43) Pub. Date: Feb. 7, 2002

(54) Postinfection human immunodeficiency virus (HIV) vaccination therapy

(75) Inventors: William J. Rutter, San Francisco, CA (US); Edward E. Penhoet, Berkeley, CA (US)

Correspondence Address: CHIRON CORPORATION, Intellectual Property -R440, P.O. Box 8097, Emeryville, CA, 94662-8097, US

(73) Assignee: CHIRON CORPORATION

(21) Appl. No.: 09/872,706

(22) Filed: Jun. 1, 2001

Related U.S. Application Data
(63) Continuation of application No. 08/917,359, filed on Aug. 26, 1997, now abandoned.
(60) Provisional application No. 60/024,575, filed on Aug. 26, 1996.

(51) Int. Cl. C07H 21/02; A61K 39/00; C12N 15/63; C07K 14/00

(52) U.S. Cl. 424/184.1; 530/351; 536/23.1; 435/320.1

(57) ABSTRACT

The invention provides for a postinfection HIV vaccination therapy having a therapeutic goal of eliminating HIV in the patient. The therapy directs administration of an agent to reduce the viral load of a patient with a measurable viral load of HIV, administration of an agent that induces an increase in production of the patient's CD4 T-cells, and administration of a vaccine capable of stimulating the patient to produce CTLs targeted to HIV-infected cells.

### FIELD OF THE INVENTION

The present invention relates to methods of treating patients infected with the human immunodeficiency viruses (HIV) by a combination antiviral, immunostimulant and vaccination therapy.

### BACKGROUND OF THE INVENTION

Recent data on HIV pathogenesis, methods to determine plasma HIV RNA, clinical trial data, and availability of new drugs point to the need for new approaches to treatment, as described in a review by an International Panel on acquired immune deficiency syndrome (AIDS), Carpenter et al, JAMA 276: 146-154 (1996). Further, the review delineates that HIV RNA measurements are important for predicting a patient's risk of clinical progression, and the review highlights a recent demonstration from clinical trials of combination therapies (referring to combinations of reverse transcriptase inhibitors) that reduction in plasma HIV RNA levels are associated with increased survival and decrease progression to AIDS. However, the review also details the problems of reverse transcriptase inhibitor therapy, including resistance to the inhibitors after time, and toxic side effects stemming from prolonged treatment. While the ultimate negative effects of these pitfalls can be delayed by the suggestions of the panel, including continued modifications of the combination of reverse transcriptase inhibitors used in a treatment for a given patient, the problem remains that amelioration of the disease by treatment with reverse transcriptase inhibitors alone is only temporary.

A new addition to the list of AIDS drugs is the HIV-protease inhibitors, which provide a new opportunity for reduction of HIV infection. However, there may be some pitfalls inherent in the use of protease inhibitors as well, including development of resistance to the protease inhibitor as described in Jacobsen et al, J. Infect. Disease, 173: 1379-1387 (1996).

Additionally, strategists for continuing AIDS antiretroviral therapy have noted that antiretroviral therapy alone may not be sufficient for immune restoration, particularly in patients with advanced disease, as described in Int'l AIDS Society-USA vol 4 (2), June 1996 pp. 16-19.

Previously described therapies that attempt to answer the need to augment an AIDS patient's immune system, including therapies that incorporate IL-2 infusions and antiretroviral therapy, are described in U.S. Pat. No. 5,419,900.

Still, elimination of HIV from an infected patient eludes the medical and research community, while data mount pointing to the virological and immunological dynamics of HIV infection, as described in Carpenter et al, JAMA 276: 146-154 (1996). These data include the early infection of lymphoid tissue as described in Pantaleo et al, Nature 362: 355-358 (1993) and Embretson et al, Nature 362: 359-362 (1993), the continuous high-level viral replication throughout the course of the disease as described in Platak et al, Science 259: 1749-1754 (1993), and Wei et al, Nature, 378: 117-122 (1995), the rapid virus population turn-over in plasma, which translates to billions of virions produced and destroyed daily, as described in Ho et al, Nature, 378: 123-126 (1995), with an estimated several billion CD4 T-cells thus produced and destroyed each day in the life of an HIV-infected individual.

There is a clear need in the medical community for design of therapies for treatment of HIV-infected individuals that can meet the clinical challenges presented by AIDS.

### SUMMARY OF THE INVENTION

One embodiment of the invention is a method of reducing human immunodeficiency virus HIV) in an HIV-infected patient, where the patient has a measurable viral load, by reducing the viral load in the patient by administering on of a first therapeutic agent, administering a second therapeutic agent capable of increasing a count of a T-cell lymphocyte expressing a cluster of differentiation-4 antigen (CD4 T-cell) in the patient, and administering a third therapeutic agent capable of increasing cytotoxic T-cell lymphocyte (CTL) number in the patient.

A further embodiment of the invention is a combination therapeutic agent for reducing HIV in an HIV-infected patient having a measurable viral load including a viral load reducer, a CD4 T-cell inducer, and a vaccine capable of increasing CTL count in the patient.

The invention relates to a method of eliminating human immunodeficiency virus (HIV) in an HIV-infected patient, the patient having a measurable viral load, comprising the steps:

(a) reducing the viral load in the patient by administration of a first therapeutic agent,

(b) administering a second therapeutic agent capable of increasing a count of a T-cell lymphocyte expressing a cluster of differentiation-4 antigen (CD4 T-cell) in the patient, and

(c) administering a third therapeutic agent capable of increasing a number of cytotoxic T-cell lymphocytes (CTLs) in the patient.

In one embodiment, the method further comprises the step (d) monitoring the patient by a diagnostic test.

In step (d), the diagnostic test can be selected from the group consisting of a cellular PCR test for a viral load, a plasma PCR test for a viral load, a cellular bDNA test for a viral load, a plasma bDNA test for a viral load, probe hybridization with HIV DNA, probe hybridization with HIV RNA, and an antibody test for detection of HIV antigen proteins.

Step (a) can comprise interrupting the life cycle of HIV in the patient

In one embodiment, interrupting the life-cycle of HIV comprises administration of the first therapeutic agent, and the first therapeutic agent is capable of inhibiting a biological interaction selected from the group consisting of a proteinprotein interaction, a protein-DNA interaction, a protein-RNA interaction, a DNA-DNA interaction, a DNA-RNA interaction, and an RNA-RNA interaction. The first therapeutic agent comprises an inhibitor selected from the group consisting of a polynucleotide, a polypeptide, an organic small molecule, a peptide, and a peptoid.

The viral load can be reduced by administration of a first therapeutic agent which comprises a therapeutic agent selected from the group consisting of a protease inhibitor, a reverse transcriptase inhibitor, an integrase inhibitor, an inhibitor of a tat/tar interaction, and an inhibitor of a rev/rre interaction.

In one embodiment, the first therapeutic agent is a protease inhibitor selected from the group consisting of Sequinivir, Indinavir, Nelfinaivir, and Ritonavir.

In another embodiment, the first therapeutic agent is a reverse transcriptase inhibitor selected from the group consisting of a nucleoside inhibitor and a non-nucleoside inhibitor. The nucleoside inhibitor comprises one selected from the group consisting of didanosine, stavudine, lamivudine, zidovudine, zalcitabine, and delavirdine.

In yet another embodiment, the first therapeutic agent comprises a combination of agents selected from the group consisting of a protease inhibitor, a reverse transcriptase inhibitor, an integrase inhibitor, an inhibitor of a tat/tar interaction and an inhibitor of a rev/rre interaction. The protease inhibitor can comprise one selected from the group consisting of Sequinivir, Indinavir, Nelfinaivir, and Ritonavir. The reverse transcriptase inhibitor can comprise one selected from the group consisting of didanosine, stavudine, lamivudine, zidovudine, zalcitabine, and delavirdine. Finally, the combination of agents can comprise a combination selected from the group consisting of a combination of zidovudine with lamivudine and Indivinavir, a combination of zidovudine and didanosine, a combination of zidovudine and zalcitabine, a combination of didanosine and stavudine, a combination of zidovudine and didanosine with a protease inhibitor, a combination of zidovudine and zalcitabine with a protease inhibitor, and a combination of didanosine and stavudine with a protease inhibitor.

In another embodiment, the first therapeutic agent can comprise an inhibitor selected from the group consisting of a polynucleotide, a polypeptide, an organic small molecule, a peptide, and a peptoid.

The combination of first therapeutic agents can comprise a therapeutic agent selected from the group consisting of a polynucleotide, a polypeptide, an organic small molecule, a peptide, and a peptoid.

In the above embodiments and other embodiments, step (b) can comprise administration of a therapeutic agent selected from the group consisting of a T-cell growth factor and a cytokine.

Specifically, the second therapeutic agent can comprise a cytokine selected from the group consisting of IL-2, IL-4, IL-7, IL-9, IL-12, IL-15, and gamma interferon (INFγ). In preferred embodiments, the cytokine can comprise an IL-2 selected from the group consisting of biologically active mature IL-2, truncated IL-2, an IL-2 variant.

In a particularly preferred embodiment, the IL-2 comprises the biologically active IL-2 variant IL-2 des Ala Ser-125.

Administration of a second therapeutic agent can comprise administration of the IL-2 by a mode selected from the group consisting of oral, parenteral, or pulmonary administration.

In these embodiments, in step (b) the cytokine is administered by administering a polynucleotide encoding the cytokine in a gene therapy protocol for expression in the patient. For example, in step (b) the IL-2 is administered by administering a polynucleotide encoding the IL-2 in a gene therapy protocol for expression in the patient. In a preferred embodiment, the gene therapy protocol comprises administration of one selected from the group consisting of naked DNA, a non-viral vector, a viral vector. In a particularly preferred embodiment, the gene therapy protocol comprises administration of a viral vector, and the viral vector comprises a retroviral vector.

Administering a second therapeutic agent capable of increasing a count of a CD4 T-cell in the patient can comprise administration of a therapeutic agent capable of inducing expression in the patient of a protein capable of increasing a count of a CD4 T-cell in a patient.

In one embodiment, the protein capable of increasing a count of a CD4 T-cell in a patient can comprise a cytokine. In a preferred embodiment, the cytokine can comprise one selected from the group consisting of IL-2, IL-4, IL-7, IL-9, IL-12, IL-15 and gamma interferon (INFγ). In a particularly preferred embodiment, the cytokine is IL-2.

In the above embodiments and other embodiments, step (c) can comprise administering a vaccine. In the above embodiments and other embodiments, step (c) can comprise administering a vaccine. The vaccine can be selected from the group consisting of a viral subunit vaccine and a nucleic acid vaccine.

In a preferred embodiment, the viral subunit vaccine comprises an HIV subunit derived from an HIV gene.

Preferably, the HIV the subunit comprises all or a portion of a protein selected from the group consisting of p24, gp41, gp120, gp160, env, rev, nef, reverse transcriptase, protease, integrase, gag, and pol subunits of an HIV gene. The subunit vaccine can comprise a fusion protein comprising at least one subunit of an HIV gene.

In a particularly preferred embodiment, the fusion protein comprises a fusion protein selected from the group consisting of a fusion of gal and pol subunits, and a fusion protein gp140 comprising a fusion of gp120 and at least a portion of gp41.

The HIV subunit can comprise an immunogenic molecule selected from the group consisting of portions of an HIV subunit, peptide derivatives of an HIV subunit, and epitopes derived from an HIV gene.

The immunogenic molecule can comprise a molecule capable of an immune response in the patient selected from the group consisting of induction of CTLs in the patient, induction of lymphocytes with T-cell helper function, and antibodies capable of neutralizing HIV.

In a preferred embodiment, the viral subunit vaccine can comprise an agent to facilitate delivery of the vaccine selected from the group consisting of a polypeptide, a peptide, a conjugate of a polypeptide and an immunogenic molecule, a conjugate of a peptide and an immunogenic molecule, a liposome, a lipid, a viral vector, and a non-viral vector. In one preferred embodiment, the agent to facilitate delivery of the vaccine can be a viral vector and can be selected from the group consisting of a retrovirus, an adenovirus, an adeno-associated virus, a herpes virus and a sindbis virus.

In another preferred embodiment, the agent to facilitate delivery of the vaccine is a non-viral vector and the non-viral vector is selected from the group consisting of naked DNA, DNA and liposomes, and particle-mediated gene transfer.

In all the embodiments described herein, administration of the vaccine can further comprise administration of an adjuvant. The adjuvant can comprise alum or an oil-in-water emulsion. The adjuvant can be an oil-in-water emulsion, and the oil-in-water emulsion can comprise a submicron oil-in-water emulsion. In a preferred embodiment, the submicron oil-in-water emulsion comprises MF59.

The vaccine can comprise a nucleic acid vaccine selected from the group consisting of a DNA vaccine, and an RNA vaccine. Administration of the nucleic acid vaccine can comprise use of an agent to facilitate delivery of the vaccine wherein the agent can be selected from the group consisting of a polypeptide, a peptide, a polysaccharide conjugate, a liposome, a lipid, a viral vector, and a non-viral vector. The agent to facilitate delivery of the vaccine can also be a viral vector selected from the group consisting of a retrovirus, an adenovirus, an adeno-associated virus, a herpes virus, an alpha virus, a semliki forest virus, and a sindbis virus. The agent to facilitate delivery of the vaccine can also be a non-viral vector and the non-viral vector can comprise one selected from the group consisting of naked DNA, DNA and liposomes, and particle-mediated gene transfer.

In one embodiment of the above methods, the nucleic acid vaccine can comprise a protein coding sequence. In another embodiment, the nucleic acid vaccine can comprise a regulatory region. The regulatory region can be selected from the group consisting of a promoter, an enhancer, a 3′ untranslated region, and a 5′ untranslated region.

In a preferred embodiment of the invention, step (a) is accomplished by administration of at least one first therapeutic agent or a combination of first therapeutic agents, step (b) is accomplished by administration of at least one second therapeutic agent, and step (c) is accomplished by administration of at least one third therapeutic agent, wherein a combined administration of the therapeutic agents of (a), (b), and (c) comprises a co-administration protocol selected from the group consisting of simultaneous administration of first, second and third therapeutic agents, sequential administration of first, second and third therapeutic agents, and administration of the first therapeutic agent or the combination of first therapeutic agents comprising step (a) followed by simultaneous administration of second and third therapeutic agents comprising steps (b) and step (c), respectively.

In one particularly preferred embodiment, step (b) comprises administration of a polypeptide T-cell growth factor and step (c) comprises immunization with a nucleic acid vaccine comprising a polynucleotide encoding all or a portion of an HIV gene. In another particularly preferred embodiment, step (b) comprises administration of a cytokine. The cytokine can be selected from the group consisting of IL-2, IL-4, IL-7, IL-9, IL-12, IL-15 and gamma interferon (INFγ). The IL-2 can comprise one selected from the group consisting of mature IL-2, an IL-2 variant, and a truncated IL-2. The IL-2 variant can be IL-2 des Ala Ser-125.

In a preferred embodiment, the subunit is selected from the group consisting all or a portion of p24, gp41, gp120, gp160, env, rev, nef, reverse transcriptase, protease, integrase, gag, and pol subunits of an HIV gene.

In another preferred embodiment of the invention, step (c) comprises a first administration of a therapeutic agent capable of increasing a number of CTLs in the patient comprising administering a vaccine selected from the group consisting of a retroviral vector, naked DNA, a polypeptide, sindbis DNA, sindbis RNA, ELVS DNA, and an adenoviral-associated vector, and a second administration comprising administering a vaccine selected from the group consisting of a retroviral vector, naked DNA, a polypeptide, sindbis DNA, sindbis RNA, ELVS DNA, and an adenoviral-associated vector.

The invention further relates to a combination therapeutic agent for eliminating HIV in an HIV-infected patient having a measurable viral load comprising a viral load reducer, a CD4 T-cell inducer, and a vaccine capable of increasing a CTL count in the patient.

In the combination therapeutic agent, the viral load reducer can comprise an agent selected from the group consisting of a protease inhibitor, a reverse transcriptase inhibitor, an integrase inhibitor, an inhibitor of a tat/tar interaction, and an inhibitor of a rev/rre interaction.

In the combination therapeutic agent, the viral load reducer can comprise a combination of therapeutic agents comprising a protease inhibitor, a reverse transcriptase inhibitor, an integrase inhibitor, an inhibitor of a tat/tar interaction, and an inhibitor of a rev/rre interaction.

In the combination therapeutic agent, the viral load reducer can comprise an agent selected from the group consisting of a polynucleotide, a polypeptide, an organic small molecule, a peptide, and a peptoid.

In the combination therapeutic agent, the CD4 T-cell inducer can comprise a cytokine selected from the group consisting of IL-2, IL-4, IL-7, IL-9, IL-12, IL-15, and gamma interferon.

In a preferred embodiment of the combination therapeutic agent, the cytokine is IL-2.

In another preferred embodiment of the combination therapeutic agent, the CD4 T-cell inducer comprises an agent selected from the group consisting of a polynucleotide, a polypeptide, an organic small molecule, a peptide, and a peptoid.

In a particularly preferred embodiment of the combination therapeutic, the CD4 T-cell inducer comprises a polynucleotide encoding a T-cell growth factor for expression in the patient.

In the combination therapeutic agent, the vaccine capable of increasing a CTL count in the patient can comprise a vaccine selected from the group consisting of a subunit vaccine and a nucleic acid vaccine.

Preferably, in the combination therapeutic agent, the subunit vaccine comprises a polypeptide selected from the group consisting of an HIV subunit, a portion of an HIV subunit, and HIV polyprotein, and a fusion of more than one HIV subunits.

Preferably, in the combination therapeutic agent, the HIV subunit comprises a subunit selected from the group consisting of p24, gp41, gp120, gp160, env, rev, nef, reverse transcriptase, protease, integrase, gag, and pol subunits of an HIV gene. In this embodiment of the combination therapeutic agent, the subunit vaccine comprises a fusion protein comprising at least one subunit of an HIV gene.

The fusion protein can comprise a fusion protein selected from the group consisting of a fusion of gal and pol, and a fusion protein gp140 comprising a fusion of gp120 and at least a portion of gp41.

Preferably, the HIV subunit can comprise an immunogenic molecule selected from the group consisting of portions of an HIV subunit, peptide derivatives of an HIV subunit, and epitopes derived from an HIV gene.

More preferably, the immunogenic molecule can comprise a molecule capable of an immune response in the patient selected from the group consisting of induction of CTLs in the patient, induction of lymphocytes with T-cell helper function, and antibodies capable of nuetralizing HIV.

In another embodiment of the combination therapeutic agent, administration of the subunit vaccine can comprise use of an agent to facilitate delivery of the vaccine, wherein the agent is selected from the group consisting of a polypeptide, a peptide, a conjugate of a polypeptide and an immunogenic molecule, a conjugate of a peptide and an immunogenic molecule, a liposome, a lipid, a viral vector, and a non-viral vector.

Preferably, the agent to facilitate delivery of the vaccine is a viral vector and the viral vector comprises one selected from the group consisting of a retrovirus, an adenovirus, an adeno-associated virus, a herpes virus and a sindbis virus.

In another preferred embodiment of the combination therapeutic agent, the agent to facilitate delivery of the vaccine is a non-viral vector and the non-viral vector comprises one selected from the group consisting of naked DNA, DNA and liposomes, and particle-mediated gene transfer.

In the embodiments of the combination therapeutic agent, the vaccine can further comprise an adjuvant. The adjuvant can comprise alum or an oil-in-water emulsion. The adjuvant can be an oil-in-water emulsion, and the oil-in-water emulsion can comprise a submicron oil-in-water emulsion.

In a preferred embodiment of the combination therapeutic agent the submicron oil-in-water emulsion comprises MF59.

In the combination therapeutic agent, the vaccine can comprise a nucleic acid vaccine selected from the group consisting of a DNA vaccine, and an RNA vaccine. In the combination therapeutic agent, the nucleic acid vaccine can comprise an agent to facilitate delivery of the vaccine selected from the group consisting of a polypeptide, a peptide, a polysaccharide conjugate, a liposome, a lipid, a viral vector, and a non-viral vector.

The combination therapeutic agent can further comprise an agent to facilitate delivery of the vaccine, wherein the agent to facilitate delivery of the vaccine is a viral vector and the viral vector can comprise one selected from the group consisting of a retrovirus, an adenovirus, an adeno-associated virus, a herpes virus and a sindbis virus.

The combination therapeutic agent can comprise an agent to facilitate delivery of the vaccine, wherein the agent to facilitate delivery of the vaccine is a non-viral vector and the non-viral vector can comprise one selected from the group consisting of naked DNA, DNA and liposomes, and particle-mediated gene transfer.

In the combination therapeutic agent, the nucleic acid vaccine can comprise a coding sequence.

In the combination therapeutic agent, the nucleic acid vaccine can comprise a regulatory region.

In a preferred embodiment of the combination therapeutic agent the regulatory region comprises one selected from the group consisting of a promoter, an enhancer, a 3′ untranslated region, and a 5′ untranslated region.

### DETAILED DESCRIPTION OF THE PREFERRED EMBODIMENTS

A method of treating HIV-infected patients has been discovered which is an aid in eliminating the virus from the patient. The method includes a protocol having several steps, including reducing the viral load of the patient, increasing the CD4 T-cells present in the patient, and increasing the patient's cytotoxic T-lymphocytes (CTLs), i.e., T-cells capable of targeting HIV-infected cells.

Definitions

“Human immunodeficiency viruses” or “HIV” refers to retroviruses that infect human CD4+ T-cells and causes acquired immunodeficiency syndrome (AIDS). HIVs are described in Fields et al, VIROLOGY (3rd Ed. Lippincott-Raven, Phil, PA 1996) Vol 2, ch. 60 pp. 1881-1952, incorporated by reference in full. Two human HiVs are known, HIV-1 and HIV-2. In addition, strains of HIV that have been identified from HIV-1 include A, B, C, D, E, F, G, H, I, O, and new strains. Further strains include strains IIIB, LAV, SF2, CM235, and US4, and others, including those described in “Human Retroviruses and AIDS”, (1995) Gerald Myers, editor, Los Alamos National Laboratory, Los Alamos N. Mex. 87545, published annually, incorporated by reference in full.

A “viral load” refers to an amount of virus in a patient, or an amount of virally infected cells in a patient. The viral load of an HIV infected patient, for example, can be a measure of infectious virus in the cells or plasma of the patient, a measure of the RNA of the virus in the cells or plasma of the patient, or a measure of proviral DNA in the infected cells of the patient, or other measures of viral RNA or proviral DNA in the patient tissues. A way to measure the viral load of a patient, can be, for example, a measure of viral RNA in the plasma, a measure of viral RNA in an infected cell, or viral DNA in an infected cell, a measure of infectious virus in the plasma, or a measure of infected cells in the blood or tissues of the patient, including lymphocyte tissues. A “measurable viral load” in a patient is that amount of virus in a patient's plasma, cells or tissue that can be measured by standard techniques. Presently, a low viral load as measured by levels of HIV RNA in plasma, is considered to be detection of about 5,000 copies of HIV RNA per mL of plasma, high levels of virus are represented in a viral load of about 30,000 to about 50,000 copies of HIV RNA per mL of plasma, and very high levels of virus are represented in a viral load of about 100,000 copies of HIV RNA per mL of plasma, as described in Carpenter et al, JAMA 276: 147-154 (1996). A viral load is presumed to exist when an amount of virus is detectable in a patient, whether in plasma, cells or tissue. It is understood that as methods of detection improve and become more sensitive, viral loads will be detectable in a patient at increasingly lower levels. Also, it is assumed that a measurable viral load is not a measure of total virus in the patient, but rather a relative measure of an amount of virus, useful for diagnosis and for monitoring the patient during a course of treatment or during progression of the disease. The amount of viral RNA, or viral DNA in plasma, cells or tissues can be measured, for example, by standard polymerase chain reaction amplification techniques (PCR) such as described in Sambrook et al. (1989), MOLECULAR CLONING: A LABORATORY MANUAL, 2d edition (Cold Spring Harbor Press, Cold Spring Harbor, N.Y.), Ausubel et al., CURRENT PROTOCOLS IN MOLECULAR BIOLOGY (1994), (Greene Publishing Associates and John Wiley & Sons, New York, N.Y.), and PCR PROTOCOLS, Cold Spring Harbor, NY 1991. An HIV quantitation method using PCR is described in WO 94/20640. Additionally, DNA probes have been used to detect HIV, as described in EP 617 132. The amount of viral RNA or viral DNA in plasma, cells or tissues can also be measured, for example, by branched DNA (bDNA) assay using such bDNA assays, for example, as described in WO 92/02526 and U.S. Pat. Nos. 5,451,503 and 4,775,619. A viral load may also be measured by probe hybridization with HIV DNA, probe hybridization with HV RNA, using standard nucleic acid hybridization techniques and an antibody test for detection of HIV antigen proteins, including for example the p24 HIV antigen.

“Administration” or “administering” as used herein refers to the process of delivering to a patient a therapeutic agent, or a combination of therapeutic agents. The process of administration can be varied, depending on the therapeutic agent, or agents, and the desired effect. For example, where several therapeutic agents are coadministered, one agent, or one combination of agents, may be delivered first, followed by a second or also a third delivery of a different therapeutic agent or several different therapeutic agents. Administration can be accomplished by any means appropriate for the therapeutic agent, for example, oral means, and parenteral means, including intravenous, subcutaneous, and intramuscular delivery, topical, mucosal, including nasal. A gene therapy protocol is considered an administration in which the therapeutic agent is a polynucleotide capable of accomplishing a therapeutic goal when expressed in the patient. A vaccination is also considered an administration, particularly in the context of administration of a therapeutic vaccination.

A “cytokine” refers to a group of secreted low molecular weight proteins that regulate the intensity and duration of an immune response by stimulating or inhibiting the proliferation of various immune cells or their secretion of antibodies or other cytokines, as described in Kuby, IMMUNOLOGY, (W.H. Freeman & Co., NY 1992). Cytokines that can increase a CD4 + T-cell count in a patient include, for example, IL2, IL-4, IL-7, IL-9, IL-12, IL-15, and gamma interferon (γINF), some of which are described in Kuby, IMMUNOLOGY (W.H., Freeman & Co., NY 1992) pp. 249 and 252-253. Some of these cytokines and others that may contribute to a biological system to result in an increase of CD4+ T-cells are also described in the following publications: IL-1, IL-2 (Karupiah et al., J. Immunology 144:290-298, 1990; Weber et al., J. Exp. Med. 166:1716-1733, 1987; Gansbacher et al., J. Exp. Med 172:1217-1224, 1990; U.S. Pat. No. 4,738,927), IL-3, IL-4 (Tepper et al., Cell 57:503-512, 1989; Golumbek et al., Science 254:713-716, 1991; U.S. Pat. No. 5,017,691), IL-5, IL-6 (Brakenhof et al., J. Immunol. 139:4116-4121, 1987; WO 90/06370), IL-7 (U.S. Pat. No. 4,965,195), IL-8, IL-9, IL-10, IL-11, IL-12, IL-13 (Cytokine Bulletin, Summer 1994), IL-14 and IL-15, particularly IL-2, IL-4, IL-6, IL-12, and IL-13, alpha interferon (Finter et al., Drugs 42(5):749-765, 1991; U.S. Pat. No. 4,892,743; U.S. Pat. No. 4,966,843; WO 85/02862; Nagata et al., Nature 284:316-320, 1980; Familletti et al., Methods in Enz. 78:387-394, 1981; Twu et al., Proc. Natl. Acad. Sci. USA 86:2046-2050, 1989; Faktor et al., Oncogene 5:867-872, 1990), beta interferon (Seif et al., J. Virol 65:664-671, 1991), gamma interferons (Radford et al., The American Society of Hepatology 20082015, 1991; Watanabe et al., PNAS 86:9456-9460, 1989; Gansbacher et al., Cancer Research 50:7820-7825, 1990; Maio et al., Can. Immunol. Immunother. 30:34-42, 1989; U.S. Pat. No. 4,762,791; U.S. Pat. No. 4,727,138), G-CSF (U.S. Pat. Nos. 4,999,291 and 4,810,643), GM-CSF (WO 85/04188), tumor necrosis factors (TNFs) (Jayaraman et al., J. Immunology 144:942-951, 1990), CD3 (Krissanen et al., Immunogenetics 26:258-266, 1987), ICAM-1 (Altman et al., Nature 338:512-514, 1989; Simmons et al., Nature 331:624-627, 1988), ICAM-2, LFA-1, LPA-3 (Wallner et al., J. Exp. Med. 166(4):923-932, 1987), MHC class I molecules, MHC class II molecules, B7.1-.3, ₂-microglobulin (Parnes et al., PNAS 78:2253-2257, 1981), chaperones such as calnexin, MHC linked transporter proteins or analogs thereof (Powis et al., Nature 354:528-531, 1991).

“Interleukin-2” or “IL-2” refers to a specific cytokine member of the interleukin family of cytokines. IL-2 is described in U.S. Pat. No. 4,569,790 to Koths et al, and IL-2 muteins, specifically the IL-2 des Ala Ser-125 is described in U.S. Reissue Pat. No. 33,653 to Mark et al. Use of IL-2 to stimulate a CD4 T-cell count in an HIV infected patient is described in U.S. Pat. No., 5,419,900 and PCT WO 94/26293.

A “CTL” is a cytotoxic T lymphocyte, and refers to a T-cell that is capable of mediating lysis of target cells following recognition of processed antigen presented on a major histocompatibility complex (MHC) molecule on the target cell, as described in Kuby, IMMUNOLOGY, (W.H. Freeman & Co., NY 1992). A CTL is responsible for searching and destroying a virally infected cell, for example, an HIV infected cell.

A “CD4 T-cell” refers to a T-cell possessing a cell membrane molecule which identifies the T lymphocyte or T-cell as a subset of lymphocytes. The cell membrane molecule is identifiable by a monoclonal antibody specific for the molecule; the antigen is called a cluster of differentiation, or CD. A CD4 T-cell expresses a caster of differentiation-4 cell surface antigen on its surface. CD4 is a cell surface glycoprotein found on a subset of the T-cells that recognize antigenic peptides complexed to class II MHC, as described in Kuby, IMMUNOLOGY, (W.H. Freeman & Co., NY 1992).

An “antigen” refers to any molecule that causes an immune response in a patient, including a cellular or a humoral immune response.

A “vaccine” refers to a preparation of an antigenic material capable of inducing an immune response against a pathogen, for example, a virus, or a virally infected cell. A vaccine can be a preventative vaccine, administered before infection, or a therapeutic vaccine administered to an infected individual. In the case of a therapeutic vaccine for HIV, the vaccine can be any vaccine capable of inducing production of CTLs in the patient where the CTLs are targeted to HIV-infected cells or HIV antigens. For example, a therapeutic vaccine for HIV treatment can be a subunit vaccine, a nucleic acid vaccine, or a whole virus vaccine.

A “subunit vaccine” refers to a therapeutic vaccine made up of something less than the whole HIV. Thus, a subunit vaccine could include polypeptide components of HIV, including, for example an HIV viral particle, or an HIV protein, or a portion of an HIV protein. Such proteins can include, for example, p24, gp41, gp120, or gp160, or variations or derivatives thereof HIV derived polypeptide components are described in EP 181 150 B 1, and U.S. Pat. No. 4,725,669. Other envelope proteins of HIV and antigens for HIV therapeutic vaccines are described in TEXT BOOK OF AIDS MEDICINE, Broder et al, ed. (Williams and Wilkins publishers, Baltimore, Md. 1994), pp. 699-711.

A “nucleic acid vaccine” refers to a vaccine derived from either RNA or DNA or also a synthetic nucleic acid designed from a viral RNA or DNA sequence. The nucleic acid can be delivered by a viral or non-viral vector or in a plasmid which includes regulatory sequences, for example, a promoter sequence which specifies transcription initiation and may be enhanced by elements 5′ of the promoter proper, a termination signal, 5′ and 3′ untranslated sequences, collectively providing for transcription of a coding region of a gene of interest. The coding region of the gene of interest can be, for example, a coding region for a polypeptide having the ability to induce production of CTL and antibodies in the patient or also a sequence of all or a portion of an HIV gene. Such a vector might also include an antibiotic resistance gene, for example, the kanamycin gene. Further, the vector might encode more than one coding region whose expression is directed by a second transcription unit or by an internal ribosome entry site (IRES) following the first gene of interest. Alternatively, the vector might encode a fusion polypeptide. Thus a nucleic acid vaccine may encode a fusion of polypeptide coding regions of distinct proteins, for example two proteins, or portions of two proteins encoded by an HIV gene. The nucleic acid vaccine may also encode T-helper peptide epitopes for stimulation of T-helper lymphocytes. Alternatively, the nucleic acid vaccine might encode two separate polypeptides, or biologically active portion of two polypeptides, the fusion polypeptide having the ability to induce CTLs production or antibody generation in a patient upon administration of the vaccine. The vector might also encode a gene whose product can augment an immune response, for example, including but not limited to GM-CSF, MCSF, interferon gamma, IL-2, or IL-3.

An “adjuvant” as used herein is defined as a substances that nonspecifically enhance or potentiate an immune response to an antigen, for example a viral pathogen.

The term “eliminating” or “eliminate” refers to reduction of the amount of HIV in a patient. This amount can be measured by any diagnostic means recognized by medical or research communities for detection and diagnosis of HIV and for monitoring the progression of disease in the patient, including, for example, measuring HIV RNA in plasma, tissues, or cells, for example, by PCR or bDNA technology. The goal of elimination or reduction of the amount of HIV in the patient is elimination of a patient's progression to clinical disease, thought to be achievable when the levels of HIV in the patient are reduced to low or undetectable levels for a reasonable period of time, and provided the immune system can return to full function during this time. Low levels of HIV in the patient, although difficult to determine in absolute numbers, can be established in relative amounts for a given patient or a patient population. For example, it is presently considered that low levels of HIV RNA when measured in the plasma are about 5,000 to about 10,000 copies of HIV RNA/mL of plasma. High levels of HIV are considered to be in a range of about 30,000 to about 50,000 copies of HIV RNA/mL of plasma, and very high levels are about 100,000 copies of HIV RNA/mL of plasma, as described in Carpenter et al, JAMA 276:146-154 (1996). As detailed in the article, these numbers do not indicate an accurate total measure of HIV in the patient, but give benchmarks for determining the level of infection in the patient. Where levels of HIV RNA are measured at different time points for a comparison, the comparison can give indications of progression or improvement in the patient. For purposes of the definition of the term “eliminating” as it refers to eliminating HIV in a patient, if a treatment can result in lowering the amount of virus to a very low or to an undetectable level, as measured, for example, by levels of HIV RNA in plasma, cells, or tissue, and this very low or undetectable level can be maintained for a reasonable period of time, it can be considered that elimination of HIV in the patient has occurred. For the purpose of the invention, where tests for measuring viral load are increasing in sensitivity, where a patient is able to maintain low levels of infection, combined with no signs of progression to clinical disease, HIV will be said to have been eliminated from the patient for all practical purposes. This is particularly true where, over the course of a reasonable period of time, the patient shows no progression to clinical disease. As the ability of the diagnostic technology improves to where previously undetected levels of HIV are detectable, an effective elimination of HIV will have been said to occur when the levels of HIV in the patient, while perhaps detectable, can be estimated to be at very low, or extremely low levels. Estimated low levels of HIV in a patient when combined with a lack of progression towards clinical disease, can be said to indicate that elimination of HIV in the patient has occurred. Likewise, where, before beginning treatment, signs of clinical disease had begun to show in a patient and when, with treatment including reduction of the patient's viral load to low levels, the patient manifests regression from clinical disease and this regression is maintained for a reasonable period of time, this is considered functional elimination of HIV from the patient. It is acknowledged that even where HIV levels fall below detectable levels in the patient, and it is considered that the patient no longer has a measurable viral load, there may still be some HIV in the patient, albeit not enough to cause the patient to manifest clinical indications of the disease.

The term a “viral load reducer” is a therapeutic agent that reduces a viral load in a patient with a measurable viral load and may be, for example a chemotherapeutic agent. Such a viral load reducer can be, for example, a protease inhibitor, or a reverse transcriptase inhibitor, or an integrase inhibitor. A viral load reducer is typically an inhibitor of a portion of the HIV life cycle that causes an arrest in the life cycle of the virus. The viral load reducer can be an inhibitor of a protein-protein, a protein-DNA, a protein-RNA, a DNA-DNA, a DNA-RNA, or an RNA-RNA interaction, where the inhibition results in arrest of the HIV life cycle. For example, an inhibitor of the tat/tar interaction or the rev/rre interaction results in reducing the active quantifiable HIV in a patient, and thus reducing the viral load of that patient. Additionally, a viral load reducer can be a combination of chemotherapeutics, for example, selected from the group of reverse transcriptase inhibitors, nucleoside or a non-nucleoside inhibitors, and protease inhibitors. Reverse transcriptase mono therapy (RT monotherapy), dual therapies, and multi-therapies can also be applied in treatment for a reduction of the viral load of a patient. RT monotherapy refers to the use of a single RT inhibitor, such as zidovudine or didanosine, while dual therapy is use of two such inhibitor, and multitherapy is use of more than two. Alternatively, dual therapy might include a nucleoside inhibitor, such as zidovudine or didanosine and a non-nucleoside inhibitor, administered in combination. Further, a combination including a protease inhibitor can be used as described. Carpenter et al, JAMA, 276:146-154 (1996).

The term “protease” as used herein refers to the viral protease. Viruses include in their makeup, proteases that serve to activate the virus by cleaving polypeptide portions of the virus necessary for the viral life cycle. Retroviral proteases are a class of aspartic proteases that are necessary for the replication of a retrovirus. The HIV-1 protease is required for infectivity of newly assembled progeny virus particles by cleaving the viral gag and gag-pol polyproteins as described in Sedlacek et al, Analytical Biochemistry 215: 306-309 (1993).

The term “protease inhibitor” as used herein is an antagonist of a target protease. The protease inhibitor can be antibody-based, a polynucleotide antagonist, a polypeptide antagonist, a peptide antagonist, or a small molecule antagonist, or derivatives or variations of these. The inhibitor is an agent that reduces the biological activity of a target protease in an in vivo or in vitro assay. In the context of treatment of HIV-infected patients, a protease inhibitor can be any agent that disables an HIV protease from activity or activation. In the context of treatment of HIV-infected patients, a protease inhibitor's effectiveness is measured by a reduction in viral load in the patient. Known protease inhibitors of HIV proteases include Sequinavir (invirase SQV) available from Hoffinan LaRoche, Indinavir (Crixivan) available from Merck Pharmaceuticals, Nelfinaivir, Viracept, and Ritonavir available from Abbott Laboratories.

“Reverse transcriptase” refers to an enzyme encoded by the HIV genome that catalyzes the synthesis of a DNA proviral molecule using a viral RNA template.

A “reverse transcriptase inhibitor” refers to any antagonist of reverse transcriptase enzymatic activity. The reverse transcriptase inhibitor can be a nucleoside or a non-nucleoside inhibitor. The reverse transcriptase inhibitor can be an antibody, a polynucleotide antagonist, a polypeptide antagonist, a peptide antagonist, or a small molecule antagonist, or derivatives or variations of these. The inhibitor is an agent that reduces the biological activity of a target reverse transcriptase in an in vivo or in vitro assay. In the context of treatment of HIV-infected patients, a reverse transcriptase inhibitor is any agent that disables an HIV reverse transcriptase from activity or activation. In the context of treatment of HIV-infected patients, a reverse transcriptase inhibitor's effectiveness is measured by a reduction in viral load in the patient. Known reverse transcriptase inhibitors of HIV proteases include nucleoside, and non-nucleoside analogue inhibitors. Nucleoside analogues include zidovudine (AZT or ZDV), didanosine (ddl), stavudine (d4T) available from Bristol Meyers Squibb, lamivudine also calledepivir (3TC) and zalcitabine (ddC). Non-nucleoside inhibitors (NNRTs) include, for example, nevirapine, lovuride (αAPA), delaviridine, HB4-097 (available from Hoechst-Bayer), and MKC442. Such inhibitors can be used in combinations with each other to increase an inhibitory effect, or to reduce a build up of resistance to the drug. For example, didanosine and stavudine can becombined, as can zidovudine and didanosine, zidovudine and lamivudine, and zidovudine/didanosine/nevirapine, as described in Int'l AIDS Society-USA, vol 4 (2) June 1996, pages 16-19, and Carpenter et al, JAMA 276: 146-154 (1996.

A “nucleic acid molecule” or a “polynucleotide,” as used herein, refers to either RNA or DNA molecule that encodes a specific amino acid sequence or its complementary strand. Nucleic acid molecules may also be non-coding sequences, for example, a ribozyme, an antisense oligonucleotide, or an untranslated portion of a gene. A “coding sequence” as used herein, refers to either RNA or DNA that encodes a specific amino acid sequence or its complementary strand. The DNA or RNA may be single stranded or double stranded. Synthetic nucleic acids or synthetic polynucleotides can be chemically synthesized nucleic acid sequences, and may also be modified with chemical moieties to render the molecule resistant to degredation. Modifications to synthetic nucleic acid molecules include nucleic acid monomers or derivative or modifications thereof, including chemical moieties. For example, phosphothioates can be used for the modification. A polynucleotide derivative can include, for example, such polynucleotides as branched DNA (bDNA). A polynucleotide can be a synthetic or recombinant polynucleotide, and can be generated, for example, by polymerase chain reaction (PCR) amplification, or recombinant expression of complementary DNA or RNA, or by chemical synthesis.

The term “an expression control sequence” or a “regulatory sequence” refers to a sequence that is conventionally used to effect expression of a gene that encodes a polypeptide and include one or more components that affect expression, including transcription and translation signals. Such a sequence includes, for example, one or more of the following: a promoter sequence, an enhancer sequence, an upstream activation sequence, a downstream termination sequence, a polyadenylation sequence, an optimal 5′ leader sequence to optimize initiation of translation in mammalian cells, and a Shine-Dalgarno sequence, a Kozak sequence, which identifies optimal residues around initiator AUG for mammalian cells. The expression control sequence that is appropriate for expression of the present polypeptide differs depending upon the host system in which the polypeptide is to be expressed. For example, in prokaryotes, such a control sequence can include one or more of a promoter sequence, a ribosomal binding site, and a transcription termination sequence. In eukaryotes, for example, such a sequence can include a promoter sequence, and a transcription termination sequence. If any necessary component of an expression control sequence is lacking in the nucleic acid molecule of the present invention, such a component can be supplied by the expression vector to effect expression. Expression control sequences suitable for use herein may be derived from a prokaryotic source, an eukaryotic source, a virus or viral vector or from a linear or circular plasmid. Further details regarding expression control sequences are provided below. An example of a regulatory sequence is the human immunodeficiency virus (“HIV-1”) promoter that is located in the U3 and R region of the HIV-1 long terminal repeat (“LTR”). Alternatively, the regulatory sequence herein can be a synthetic sequence, for example, one made by combining the UAS of one gene with the remainder of a requisite promoter from another gene, such as the GADP/ADH2 hybrid promoter.

Any “polypeptide” of the invention includes any part of the protein including the mature protein, and further include truncations, variants, alleles, analogs and derivatives thereof Variants can be spliced variants expressed from the same gene as the related protein. Unless specifically mentioned otherwise, such a polypeptide possesses one or more of the bioactivities of the protein, including for example protease activity, or inhibition of a protease. This term is not limited to a specific length of the product of the gene. Thus, polypeptides that are identical or contain at least 60%, preferably 70%, more preferably 80%, and most preferably 90% homology to the target protein or the mature protein, wherever derived, from human or nonhuman sources are included within this definition of a polypeptide. Also included, therefore, are alleles and variants of the product of the gene that contain amino acid substitutions, deletions, or insertions. The amino acid substitutions can be conservative amino acid substitutions or substitutions to eliminate non-essential amino acid residues such as to alter a glycosylation site, a phosphorylation site, an acetylation site, or to alter the folding pattern by altering the position of the cysteine residue that is not necessary for function, etc. Conservative amino acid substitutions are those that preserve the general charge, hydrophobicity/hydrophilicity and/or steric bulk of the amino acid substituted, for example, substitutions between the members of the following groups are conservative substitutions: Gly/Ala, Val/Ile/Leu, Asp/Glu, Lys/Arg, Asn/Gln, Ser/Cys/Thr and Phe/Trp/Tyr. Analogs include peptides having one or more peptide mimics, also known as peptoids, that possess the target protein-like activity. Included within the definition are, for example, polypeptides containing one or more analogs of an amino acid (including, for example, unnatural amino acids, etc.), polypeptides with substituted linkages, as well as other modifications known in the art, both naturally occurring and nonnaturally occurring. The term “polypeptide” also does not exclude post-expression modifications of the polypeptide, for example, glycosylations, acetylations, phosphorylations, myristoylations and the like.

The term “fusion protein” or “fusion polypeptide” refers to the recombinant expression of more than one heterologous coding sequence in a vector such that expression of the polypeptide in the vector results in expression of one polypeptide that includes more than one protein or portion of more than one protein. Fusion proteins can be called chimeric proteins. Most optimally, the fusion protein retains the biological activity of the polypeptide units from which it is built, and preferably, the fusion protein generates a synergistic improved biological activity by combining the portion of the separate proteins to form a single polypeptide. Examples of fusion proteins useful for the invention include the gag/pol fusion protein and a fusion protein called gp140 that includes gp120 and a portion of gp41.

The term “inhibitory amount” as used herein refers to that amount that is effective for production of inhibition of a protein that has biological activity, including for example inhibition of a protease, a reverse transcriptase, an integrase, or a biological interaction involving two or more molecules. In a therapeutic context, the precise inhibitory amount of an inhibitor varies depending upon the health and physical condition of the individual to be treated, the capacity of the individual's ability to adjust to the change in metabolism and body size, the formulation, and the attending physician's assessment of the medical situation, and other relevant factors. It is expected that the amount will fall in a relatively broad range that can be determined through routine trials. A sufficient amount of an inhibitor will be that amount capable of effecting an inhibition of HIV, or an activity of HIV.

A “therapeutically effective amount” is that amount that will generate the desired therapeutic outcome. For example, if the therapeutic effect desired is reduction of a viral load the amount will be the amount of a viral load reducing agent, or combination of agents that reduce a patient's measurable viral load. Where the therapeutic effect is a stimulation of an immune response in the patient, for example, stimulation of production of CD4 T-cells in a patient, the effective amount of an agent to accomplish this in the patient will be that amount that results in a stimulation of CD4 T-cells in the patient. Similarly, where the desired therapeutic effect is stimulation of CTLs specific for HIV-infected, the effective amount of the therapeutic agent will be that amount that accomplishes stimulation of CTLs capable of targeting a patient's HIV-infected cells.

A “therapeutic agent” as used herein can be any agent that accomplishes one or more of the therapeutic elements of the invention. For example, where the therapeutic agent is one designed to reduce the viral load of an HIV-infected patient, the therapeutic agent can be a single agent or a combination of agents, for example, a combination of more than one protease inhibitors, or a combination of more than one protease inhibitor in combination with a reverse transcriptase inhibitor. Optimally, a therapeutic agent will achieve, alone or in combination with other agents a therapeutic goal. Thus, for example, the therapeutic agent used for reducing the viral load in the patient may be a combination of agents each of which reduces the viral load of the patient, but when used together reduces the viral load of the patient to a lower level, or with greater speed, or with the added benefit either of increased long term maintenance of the reduced viral load in the patient, or reduced toxicity. These therapeutic agents can be for example, a small organic molecule, a peptide, a peptoid, a polynucleotide, a polypeptide, or a nucleoside. Also by example, with regard to the therapeutic element of the invention that involves stimulating CD4 T-cell production in the HIV-infected patient, the therapeutic agent that accomplishes this stimulation in the patient can be any agent that functions to do so, for example, a small molecule, a peptide, a peptoid, a polynucleotide, or a polypeptide. Where that agent is, for example, IL-2, the IL-2 can be a polypeptide form of IL-2, a derivative or variant of IL-2 polypeptide, a polynucleotide encoding all or a portion of an IL-2 polypeptide, or all or a portion of an IL-2 polypeptide derivative or variant, a small molecule mimic of IL-2 activity, a peptide mimic of IL-2, a peptoid mimic of IL-2 activity, or an agent capable of inducing the endogenous production of IL-2 in the patient thus inducing the required effect of stimulating CD4 T-cells in the patient. Where the therapeutic agent is designed to stimulate the production of CTLs in the patient, that agent can be, for example, a vaccine, and the vaccine can be, for example, a virus subunit vaccine, or a nucleic acid vaccine.

A “combination therapeutic agent” is a therapeutic composition having several components that produce when administered together their separate effects. The separate effects of the combination therapeutic agent combine to result in a larger therapeutic effect, for example recovery from disease and long term survival. An example of separate effects resulting from administration of a combination therapeutic agent is the combination of such effects as viral load reduction, an increase in CD4 T-cells, and an increase in CTLs targeting HIV-infected cells.

The term “binding pair” refers to a pair of molecules capable of a binding interaction between the two molecules. Usually a binding interaction furthers a cell signal or cellular event. The term binding pair can refer to a protein/protein, protein-DNA, protein-RNA, DNA-DNA, DNA-RNA, and RNA-RNA binding interactions, and can also include a binding interaction between a small molecule, a peptoid, or a peptide and a protein, DNA, or RNA molecule, in which the components of the pair bind specifically to each other with a higher affinity than to a random molecule, such that upon binding, for example, in case of a ligand/receptor interaction, the binding pair triggers a cellular or an intercellular response. An example of a ligand/receptor binding pair is a pair formed between PDGF (platelet derived growth factor) and a PDGF receptor. An example of a different binding pair is an antigen/antibody pair in which the antibody is generated by immunization of a host with the antigen. Another example of a binding pair is the formation of a binding pair between a protease and a protease inhibitor, or a protease substrate and a protease inhibitor. Specific binding indicates a binding interaction having a low dissociation constant, which distinguishes specific binding from non-specific, background binding. Specific binding is characterized by at least 5, 10, or 20-fold higher binding then to non-specific background components. Inhibition of a biological interaction can be accomplished by inhibiting an in vivo binding interaction such as, for example, a DNA-protein interaction. Such inhibition can be accomplished, for example, by an inhibitor that bind the protein, or by an inhibitor that binds the DNA, in either case, thus preventing the original endogenous binding interaction, and so the biological activity that follows from it.

The term “pharmaceutically acceptable carrier” refers to a carrier for administration of a therapeutic agent, such as, for example, a polypeptide, polynucleotide, small molecule, peptoid, or peptide, refers to any pharmaceutically acceptable carrier that does not itself induce the production of antibodies harmful to the individual receiving the composition, and which may be administered without undue toxicity.

The term “small molecule” as used herein refers to an organic molecule derived, for example, from a small molecule library.

The term “peptide” and the term “peptoid” as used herein refers to a peptide or peptoid (a peptide derivative) derived, for example, from a peptide library.

The term subunit, as used herein refers to anything less than the whole virus, such as polypeptides of HIV, as described in Fields et al, VIROLOGY (3rd Ed. Lippincott-Raven Phil, Pa. 1996) vol 2, ch. 60 (pp. 1881-1952). The subunits and polyprotein precursors can be useful for generation of a subunit vaccine for stimulating CTLs in an HIV-infected patient, include but are not limited to gag, pol, and env, and the DNA or RNA encoding the same.

Therapeutic agents of the invention, including for example subunit vaccines, nucleic acid vaccines, and a polynucleotide, polypeptide, or peptide therapeutic agents can be made using the following exemplary expression systems. Below are some exemplary expression systems in bacteria, yeast, insects, amphibians, and mammals.

Additionally, variations of any polynucleotide or polypeptide can be made by conventional techniques, including PCR or site-directed mutagenesis. The DNA construct so synthesized can be ligated to an expression plasmid containing an appropriate promoter for expression in a desired host expression system. Expression plasmids with various promoters are currently available commercially. Further exemplary details regarding expression systems are provided below.

Expression Systems

Although the methodology described below is believed to contain sufficient details to enable one skilled in the art to practice the present invention, other items not specifically exemplified, such as plasmids, can be constructed and purified using standard recombinant DNA techniques described in, for example, Sambrook et al. (1989), MOLECULAR CLONING: A LABORATORY MANUAL, 2d edition (Cold Spring Harbor Press, Cold Spring Harbor, N.Y.), and Ausubel et al., CURRENT PROTOCOLS IN MOLECULAR BIOLOGY (1994), (Greene Publishing Associates and John Wiley & Sons, New York, N.Y.). under the current regulations described in United States Dept. of HEW, NATIONAL INSTITUTE OF HEALTH (NIH) GUIDELINES FOR RECOMBINANT DNA RESEARCH. These references include procedures for the following standard methods: cloning procedures with plasmids, transformation of host cells, cell culture, plasmid DNA purification, phenol extraction of DNA, ethanol precipitation of DNA, agarose gel electrophoresis, purification of DNA fragments from agarose gels, and restriction endonuclease and other DNA-modifying enzyme reactions.

Expression in Bacterial Cells

Control elements for use in bacteria include promoters, optionally containing operator sequences, and ribosome binding sites. Useful promoters include sequences derived from sugar metabolizing enzymes, such as galactose, lactose (lac) and maltose. Additional examples include promoter sequences derived from biosynthetic enzymes such as tryptophan (trp), the β-lactamase (bla) promoter system, bacteriophage λPL, and T7. In addition, synthetic promoters can be used, such as the tac promoter. The β-lactamase and lactose promoter systems are described in Chang et al., Nature (1978) 275: 615, and Goeddel et al., Nature (1979) 281: 544; the alkaline phosphatase, tryptophan (trp) promoter system are described in Goeddel et al., Nucleic Acids Res. (1980) 8: 4057 and EP 36,776 and hybrid promoters such as the tac promoter is described in U.S. Pat. No. 4,551,433 and de Boer et al., Proc. Natl. Acad. Sci. USA (1983) 80: 21-25. However, other known bacterial promoters useful for expression of eukaryotic proteins are also suitable. A person skilled in the art would be able to operably ligate such promoters to the coding sequences of interest, for example, as described in Siebenlist et al., Cell (1980) 20: 269, using linkers or adaptors to supply any required restriction sites. Promoters for use in bacterial systems also generally will contain a Shine-Dalgarno (SD) sequence operably linked to the DNA encoding the target polypeptide. For prokaryotic host cells that do not recognize and process the native target polypeptide signal sequence, the signal sequence can be substituted by a prokaryotic signal sequence selected, for example, from the group of the alkaline phosphatase, penicillinase, Ipp, or heat stable enterotoxin II leaders. The origin of replication from the plasmid pBR322 is suitable for most Gram-negative bacteria.

The foregoing systems are particularly compatible with Escherichia coli. However, numerous other systems for use in bacterial hosts including Gram-negative or Gram-positive organisms such as Bacillus spp., Streptococcus spp., Streptomyces spp., Pseudomonas species such as P. aeruginosa, Salmonella typhimurium, or Serratia marcescans, among others. Methods for introducing exogenous DNA into these hosts typically include the use of CaCl₂ or other agents, such as divalent cations and DMSO. DNA can also be introduced into bacterial cells by electroporation, nuclear injection, or protoplast fusion as described generally in Sambrook et al. (1989), cited above. These examples are illustrative rather than limiting. Preferably, the host cell should secrete minimal amounts of proteolytic enzymes. Alternatively, in vitro methods of cloning, e.g., PCR or other nucleic acid polymerase reactions, are suitable.

Prokaryotic cells used to produce the target polypeptide of this invention are cultured in suitable media, as described generally in Sambrook et al., cited above.

Expression in Yeast Cells

Expression and transformation vectors, either extrachromosomal replicons or integrating vectors, have been developed for transformation into many yeasts. For example, expression vectors have been developed for, among others, the following yeasts: Saccharomyces cerevisiae, as described in Hinnen et al., Proc. Natl. Acad Sci. USA (1978) 75: 1929; Ito et al., J. Bacteriol. (1983) 153: 163; Candida albicans as described in Kurtz et al., Mol. Cell. Biol. (1986) 6: 142; Candida maltosa, as described in Kunze et al., J. Basic Microbiol. (1985) 25: 141; Hansenula polymorpha, as described in Gleeson etal., J. Gen. Microbiol. (1986) 132: 3459 and Roggenkamp et al., Mol. Gen. Genet. (1986) 202:302); Kluyveromyces fragilis, as described in Das et al., J. Bacteriol. (1984) 158: 1165; Kluyveromyces lactis, as described in De Louvencourt et al., J. Bacteriol. (1983) 154: 737 and Van den Berg et al., Bio/Technology (1990) 8: 135; Pichia guillerimondii, as described in Kunze et al., J. Basic Microbiol. (1985) 25: 141; Pichia pastoris, as described in Cregg et al., Mol. Cell. Biol. (1985) 5: 3376 and U.S. Pat. Nos. 4,837,148 and 4,929,555; Schizosaccharomyces pombe, as described in Beach and Nurse, Nature (1981) 300: 706; and Yarrowia lipolytica, as described in Davidow et al., Curr. Genet. (1985) 10: 380 and Gaillardin et al., Curr. Genet. (1985) 10: 49, Aspergillus hosts such as A. nidulans, as described in Ballance et al., Biochem. Biophys. Res. Commun. (1983) 112: 284-289; Tilburn et al., Gene (1983) 26: 205-221 and Yelton et al., Proc. Natl. Acad. Sci. USA (1984) 81: 1470-1474, and A. niger, as described in Kelly and Hynes, EMBO J. (1985) 4: 475479; Trichoderma reesia, as described in EP 244,234, and filamentous fungi such as, e.g, Neurospora, Penicillium, Tolypocladium, as described in WO 91/00357.

Control sequences for yeast vectors are known and include promoters regions from genes such as alcohol dehydrogenase (ADH), as described in EP 284,044, enolase, glucokinase, glucose-6-phosphate isomerase, glyceraldehyde-3-phosphatedehydrogenase (GAP or GAPDH), hexokinase, phosphofructokinase, 3phosphoglycerate mutase, and pyruvate kinase (PyK), as described in EP 329,203. The yeast PHO5 gene, encoding acid phosphatase, also provides useful promoter sequences, as described in Myanohara et al., Proc. Natl. Acad. Sci. USA (1983) 80: 1. Other suitable promoter sequences for use with yeast hosts include the promoters for 3-phosphoglycerate kinase, as described in Hitzeman et al., J. Biol. Chem. (1980) 255: 2073, or other glycolytic enzymes, such as pyruvate decarboxylase, triosephosphate isomerase, and phosphoglucose isomerase, as described in Hess et al., J. Adv. Enzyme Reg. (1968) 7: 149 and Holland et al., Biochemistry (1978) 17:4900. Inducible yeast promoters having the additional advantage of transcription controlled by growth conditions, include from the list above and others the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in Hitzeman, EP 073,657. Yeast enhancers also are advantageously used with yeast promoters. In addition, synthetic promoters which do not occur in nature also function as yeast promoters. For example, upstream activating sequences (UAS) of one yeast promoter may be joined with the transcription activation region of another yeast promoter, creating a synthetic hybrid promoter. Examples of such hybrid promoters include the ADH regulatory sequence linked to the GAP transcription activation region, as described in U.S. Pat. Nos. 4,876,197 and 4,880,734. Other examples of hybrid promoters include promoters which consist of the regulatory sequences of either the ADH2, GAL4, GAL10, or PHO5 genes, combined with the transcriptional activation region of a glycolytic enzyme gene such as GAP or PyK, as described in EP 164,556. Furthermore, a yeast promoter can include naturally occurring promoters of non-yeast origin that have the ability to bind yeast RNA polymerase and initiate transcription.

Other control elements which may be included in the yeast expression vectors are terminators, for example, from GAPDH and from the enolase gene, as described in Holland et al., J. Biol. Chem. (1981) 256: 1385, and leader sequences which encode signal sequences for secretion. DNA encoding suitable signal sequences can be derived from genes for secreted yeast proteins, such as the yeast invertase gene as described in EP 012,873 and JP 62,096,086 and the a-factor gene, as described in U.S. Pat. Nos. 4,588,684, 4,546,083 and 4,870,008; EP 324,274; and WO 89/02463. Alternatively, leaders of non-yeast origin, such as an interferon leader, also provide for secretion in yeast, as described in EP 060,057.

Methods of introducing exogenous DNA into yeast hosts are well known in the art, and typically include either the transformation of spheroplasts or of intact yeast cells treated with alkali cations.

Transformations into yeast can be carried out according to the method described in Van Solingen et al., J. Bact. (1977) 130:946 and Hsiao et al., Proc. Natl. Acad Sci. (USA) (1979) 76:3829. However, other methods for introducing DNA into cells such as by nuclear injection, electroporation, or protoplast fusion may also be used as described generally in Sambrook et al., cited above.

For yeast secretion the native target polypeptide signal sequence may be substituted by the yeast invertase, α-factor, or acid phosphatase leaders. The origin of replication from the 2μ plasmid origin is suitable for yeast. A suitable selection gene for use in yeast is the trpl gene present in the yeast plasmid described in Kingsman et al., Gene (1979) 7: 141 or Tschemper et al., Gene (1980) 10:157. The trp1 gene provides a selection marker for a mutant strain of yeast lacking the ability to grow in tryptophan. Similarly, Leu2-deficient yeast strains (ATCC 20,622 or 38,626) are complemented by known plasmids bearing the Leu2 Gene.

For intracellular production of the present polypeptides in yeast, a sequence encoding a yeast protein can be linked to a coding sequence of the polypeptide to produce a fusion protein that can be cleaved intracellularly by the yeast cells upon expression. An example, of such a yeast leader sequence is the yeast ubiquitin gene.

Expression in Insect Cells

Baculovirus expression vectors (BEVs) are recombinant insect viruses in which the coding sequence for a foreign gene to be expressed is inserted behind a baculovirus promoter in place of a viral gene, e.g., polyhedrin, as described in Smith and Summers, U.S. Pat. No., 4,745,051.

An expression construct herein includes a DNA vector useful as an intermediate for the infection or transformation of an insect cell system, the vector generally containing DNA coding for a baculovirus transcriptional promoter, optionally but preferably, followed downstream by an insect signal DNA sequence capable of directing secretion of a desired protein, and a site for insertion of the foreign gene encoding the foreign protein, the signal DNA sequence and the foreign gene being placed under the transcriptional control of a baculovirus promoter, the foreign gene herein being the coding sequence of the polypeptide.

The promoter for use herein can be a baculovirus transcriptional promoter region derived from any of the over 500 baculoviruses generally infecting insects, such as, for example, the Orders Lepidoptera, Diptera, Orthoptera, Coleoptera and Hymenoptera including, for example, but not limited to the viral DNAs of Autographo californica MNPV, Bombyx mori NPV, rrichoplusia ni MNPV, Rachiplusia ou MNPV or Galleria mellonella MNPV. Thus, the baculovirus transcriptional promoter can be, for example, a baculovirus immediate-early gene IEI or IEN promoter; an immediateearly gene in combination with a baculovirus delayed-early gene promoter region selected from the group consisting of a 39K and a HindIII fragment containing a delayed-early gene; or a baculovirus late gene promoter. The immediate-early or delayed-early promoters can be enhanced with transcriptional enhancer cements.

Particularly suitable for use herein is the strong polyhedrin promoter of the baculovirus, which directs a high level of expression of a DNA insert, as described in Friesen et al. (1986) “The Regulation of Baculovirus Gene Expression” in: THE MOLECULAR BIOLOGY OF BACULOVIRUSES (W.Doerfler, ed.); EP 127,839 and EP 155,476; and the promoter from the gene encoding the p10 protein, as described in Vlak et al., J. Gen. Virol. (1988) 69:765-776.

The plasmid for use herein usually also contains the polyhedrin polyadenylation signal, as described in Miller et al., Ann. Rev. Microbiol. (1988) 42:177 and a procaryotic ampicillin-resistance (amp) gene and an origin of replication for selection and propagation in E. coli. DNA encoding suitable signal sequences can also be included and is generally derived from genes for secreted insect or baculovirus proteins, such as the baculovirus polyhedrin gene, as described in Carbonell et al., Gene (1988) 73:409, as well as mammalian signal sequences such as those derived from genes encoding human a-interferon as described in Maeda et al., Nature (1985) 315:592-594; human gastrin-releasing peptide, as described in Lebacq-Verheyden et al., Mol. Cell. Biol. (1988) 8: 3129; human IL-2, as described in Smith et al., Proc. Natl. Acad Sci. USA (1985) 82:8404; mouse IL-3, as described in Miyajima et al., Gene (1987) 58:273; and human glucocerebrosidase, as described in Martin et al., DNA (1988) 7:99.

Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori host cells have been identified and can be used herein. See, for example, the description in Luckow et al., Bio/Technology(1988) 6: 47-55, Miller et al., in GENETIC ENGINEERING (Setlow, J. K. et al. eds.), Vol. 8 (Plenum Publishing, 1986), pp. 277-279, and Maeda et al., Nature, (1985) 315: 592-594. A variety of such viral strains are publicly available, e.g., the L-1 variant of Autographa californica NPV and the Bm-5 strain of Bombyx mori NPV. Such viruses may be used as the virus for transfection of host cells such as Spodoptera frugiperda cells.

Other baculovirus genes in addition to the polyhedrin promoter may be employed to advantage in a baculovirus expression system. These include immediate-early (alpha), delayed-early (beta), late (gamma), or very late (delta), according to the phase of the viral infection during which they are expressed. The expression of these genes occurs sequentially, probably as the result of a “cascade” mechanism of transcriptional regulation. Thus, the immediate-early genes are expressed immediately after infection, in the absence of other viral functions, and one or more of the resulting gene products induces transcription of the delayed-early genes. Some delayed-early gene products, in turn, induce transcription of late genes, and finally, the very late genes are expressed under the control of previously expressed gene products from one or more of the earlier classes. One relatively well defined component of this regulatory cascade is IEI, a preferred immediate-early gene of Autographo californica nuclear polyhedrosis virus (AcMNPV). IEI is pressed in the absence of other viral functions and encodes a product that stimulates the transcription of several genes of the delayed-early class, including the preferred 39K gene, as described in Guarino and Summers, J. Virol. (1986) 57:563-571 and J. Virol. (1987) 61:2091-2099 as well as late genes, as described in Guanno and Summers, Virol. (1988) 162:444-451.

Immediate-early genes as described above can be used in combination with a baculovirus gene promoter region of the delayed-early category. Unlike the immediate-early genes, such delayed-early genes require the presence of other viral genes or gene products such as those of the immediate-early genes. The combination of immediate-early genes can be made with any of several delayed-early gene promoter regions such as 39K or one of the delayed-early gene promoters found on the HindIII fragment of the baculovirus genome. In the present instance, the 39 K promoter region can be linked to the foreign gene to be expressed such that expression can be further controlled by the presence of IEI, as described in L. A. Guarino and Summers (1986a), cited above; Guarino & Summers (1986b) J. Virol., (1986) 60:215-223, and Guarino et al. (1986c), J. Virol. (1986) 60:224-229.

Additionally, when a combination of immediate-early genes with a delayed-early gene promoter region is used, enhancement of the expression of heterologous genes can be realized by the presence of an enhancer sequence in direct cis linkage with the delayed-early gene promoter region. Such enhancer sequences are characterized by their enhancement of delayed-early gene expression in situations where the immediate-early gene or its product is limited. For example, the hr5 enhancer sequence can be linked directly, in cis, to the delayed-early gene promoter region, 39K, thereby enhancing the expression of the cloned heterologous DNA as described in Guarino and Summers (1986a), (1986b), and Guarino et al. (1986).

The polyhedrin gene is classified as a very late gene. Therefore, transcription from the polyhedrin promoter requires the previous expression of an unknown, but probably large number of other viral and cellular gene products. Because of this delayed expression of the polyhedrin promoter, state-of-the-art BEVs, such as the exemplary BEV system described by Smith and Summers in, for example, U.S. Pat. No., 4,745,051 will express foreign genes only as a result of gene expression from the rest of the viral genome, and only after the viral infection is well underway. This represents a limitation to the use of existing BEVs. The ability of the host cell to process newly synthesized proteins decreases as the baculovirus infection progresses. Thus, gene expression from the polyhedrin promoter occurs at a time when the host cell's ability to process newly synthesized proteins is potentially diminished for certain proteins such as human tissue plasminogen activator. As a consequence, the expression of secretory glycoproteins in BEV systems is complicated due to incomplete secretion of the cloned gene product, thereby trapping the cloned gene product within the cell in an incompletely processed form.

While it has been recognized that an insect signal sequence can be used to express a foreign protein that can be cleaved to produce a mature protein, the present invention is preferably practiced with a mammalian signal sequence appropriate for the gene expressed.

An exemplary insect signal sequence suitable herein is the sequence encoding for a Lepidopteran adipokinetic hormone (AKH) peptide. The AKH family consists of short blocked neuropeptides that regulate energy substrate mobilization and metabolism in insects. In a preferred embodiment, a DNA sequence coding for a Lepidopteran Manduca sexta AKH signal peptide can be used. Other insect AKH signal peptides, such as those from the Orthoptera Schistocerca gregaria locus can also be employed to advantage. Another exemplary insect signal sequence is the sequence coding for Drosophila cuticle proteins such as CPI, CP2, CP3 or CP4.

Currently, the most commonly used transfer vector that can be used herein for introducing foreign genes into AcNPV is pAc373. Many other vectors, known to those of skill in the art, can also be used herein. Materials and methods for baculovirus/insect cell expression systems are commercially available in a kit form from companies such as Invitrogen (San Diego Calif.) (“Maxdac” kit). The techniques utilized herein are generally known to those skilled in the art and are fully described in Summers and Smith, A MANUAL OF METHODS FOR BACULOVIRUS VECTORS AND INSECT CELL CULTURE PROCEDURES, Texas Agricultural Experiment Station Bulletin No. 1555, Texas A&M University (1987); Smith et al., Mol. Cell. Biol. (1983) 3: 2156, and Luckow and Summers, Virology (1989) 17:31. These include, for example, the use of pVL985 which alters the polyhedrin start codon from ATG to ATT, and which introduces a BamHI cloning site 32 basepairs downstream from the ATT, as described in Luckow and Summers, Virology (1989) 17:31.

Thus, for example, for insect cell expression of the present polypeptides, the desired DNA sequence can be inserted into the transfer vector, using known techniques. An insect cell host can be cotransformed with the transfer vector containing the inserted desired DNA together with the genomic DNA of wild type baculovirus, usually by cotransfection. The vector and viral genome are allowed to recombine resulting in a recombinant virus that can be easily identified and purified. The packaged recombinant virus can be used to infect insect host cells to express a desired polypeptide.

Other methods that are applicable herein are the standard methods of insect cell culture, cotransfection and preparation of plasmids are set forth in Summers and Smith (1987), cited above. This reference also pertains to the standard methods of cloning genes into AcMNPV transfer vectors, plasmid DNA isolation, transferring genes into the AcmMNPV genome, viral DNA purification, radiolabeling recombinant proteins and preparation of insect cell culture media. The procedure for the cultivation of viruses and cells are described in Volkman and Summers, J. Virol. (1975) 19:820-832 and Volkman, et al, J. Virol. (1976)19:820-832.

Expression in Amphibian Cells

Expression of libraries of candidates for the practice of the invention can be conducted in the oocytes of amphibians. One amphibian particularly useful for this purpose is Xenopus laevis because of the capacity of the oocytes of this animal to express large libraries. Expression systems for X. laevis and other amphibians is established and expression conducted as described in Lustig and Kirschner, PNAS (1995) 92: 6234-38, Krieg and Melton (1987) Meth Enzymol 155:397-415 and Richardson et al. (1988) Bio/Technology 6:565-570.

For construction of libraries using Xenopus laevis oocytes, Xenopus oocytes are injected with cRNA libraries of candidate factors. The cRNA libraries are from plasmid DNAs from small cDNA library pools from a source such as a cell line or an animal organ. The plasmid DNAs are in vitro transcribed to cRNA and then injected into the oocyte, as described in Lustig and Kirschner, Krieg and Melton and Richardson et al, cited previously. The oocyte is incubated overnight at 18° C. The next day the oocyte is placed in microwells in contact with responsive cells. The microwells are incubated at 37° C. for 30 minutes to 3 hours. Candidate stimulatory or inhibitory factors, ligands, antagonists, or transcription factors are then expressed and secreted by the oocytes.

Expression in Mammalian Cells

Typical promoters for mammalian cell expression of the polypeptides of the invention include the SV40 early promoter, the CMV promoter, the mouse mammary tumor virus LTR promoter, the adenovirus major late promoter (Ad MLP), and the herpes simplex virus promoter, among others. Other non-viral promoters, such as a promoter derived from the murine metallothionein gene, will also find use in mammalian constructs. Mammalian expression may be either constitutive or regulated (inducible), depending on the promoter. Typically, transcription termination and polyadenylation sequences will also be present, located 3′ to the translation stop codon. Preferably, a sequence for optimization of initiation of translation, located 5′ to the polypeptide coding sequence, is also present. Examples of transcription terminator/polyadenylation signals include those derived from SV40, as described in Sambrook et al. (1989), cited previously. Introns, containing splice donor and acceptor sites, may also be designed into the constructs of the present invention.

Enhancer elements can also be used herein to increase expression levels of the mammalian constructs. Examples include the SV40 early gene enhancer, as described in Dijkema et al., EMBO J. (1985) 4:761 and the enhancer/promoter derived from the long terminal repeat (LTR) of the Rous Sarcoma Virus, as described in Gorman et al., Proc. Natl. Acad Sci. USA (1982b) 79:6777 and human cytomegalovirus, as described in Boshart et al., Cell (1985) 41:521. A leader sequence can also be present which includes a sequence encoding a signal peptide, to provide for the secretion of the foreign protein in mammalian cells. Preferably, there are processing sites encoded between the leader fragment and the gene of interest such that the leader sequence can be cleaved either in vivo or in vitro. The adenovirus tripartite leader is an example of a leader sequence that provides for secretion of a foreign protein in mammalian cells.

Once complete, the mammalian expression vectors can be used to transform any of several mammalian cells. Methods for introduction of heterologous polynucleotides into mammalian cells are known in the art and include dextran-mediated transfection, calcium phosphate precipitation, polybrene mediated transfection, protoplast fusion, electroporation, encapsulation of the polynucleotide(s) in liposomes, and direct microinjection of the DNA into nuclei. General aspects of mammalian cell host system transformations have been described by Axel in U.S. Pat. No. 4,399,216.

Therapeutic agents of the invention can include organic small molecules, peptides and peptoids that antagonize a target polypeptide activity, a target polynucleotide, or that facilitate a desired biological activity in a patient. Examplary synthesis of some small molecule libraries are described below.

Small Molecule Library Synthesis

Small molecule libraries are made as follows. A “library” of peptides may be synthesized and used following the methods disclosed in U.S. Pat. No. 5,010,175, (the '175 patent) and in PCT WO91/17823. In method of the '175 patent, a suitable peptide synthesis support, for example, a resin, is coupled to a mixture of appropriately protected, activated amino acids.

The method described in W091/17823 is similar. However, instead of reacting the synthesis resin with a mixture of activated amino acids, the resin is divided into twenty equal portions, or into a number of portions corresponding to the number of different amino acids to be added in that step, and each amino acid is coupled individually to its portion of resin. The resin portions are then combined, mixed, and again divided into a number of equal portions for reaction with the second amino acid. Additionally, one may maintain separate “subpools” by treating portions in parallel, rather than combining all resins at each step. This simplifies the process of determining which peptides are responsible for any observed alteration of gene expression in a responsive cell.

The methods described in W091/17823 and U.S. Pat. No. 5,194,392 enable the preparation of such pools and subpools by automated techniques in parallel, such that all synthesis and resynthesis may be performed in a matter of days.

Further alternative agents include small molecules, including peptide analogs and derivatives, that can act as stimulators or inhibitors of gene expression, or as ligands or antagonists. Some general means contemplated for the production of peptides, analogs or derivatives are outlined in CHEMISTRY AND BIOCHEMISTRY OF AMINO ACIDS, PEPTIDES, AND PROTEINS—A SURVEY OF RECENT DEVELOPMENTS, Weinstein, B. ed., Marcell Dekker, Inc., publ. New York (1983). Moreover, substitution of D-amino acids for the normal L-stereoisomer can be carried out to increase the half-life of the molecule.

Peptoids, polymers comprised of monomer units of at least some substituted amino acids, can act as small molecule stimulators or inhibitors herein and can be synthesized as described in PCT 91/19735. Presently preferred amino acid substitutes are N-alkylated derivatives of glycine, which are easily synthesized and incorporated into polypeptide chains. However, any monomer units which allow for the sequence specific synthesis of pools of diverse molecules are appropriate for use in producing peptoid molecules. The benefits of these molecules for the purpose of the invention is that they occupy different conformational space than a peptide and as such are more resistant to the action of proteases.

Peptoids are easily synthesized by standard chemical methods. The preferred method of synthesis is the “submonomer” technique described by R. Zuckermann et al., J. Am. Chem. Soc. (1992) 114:10646-7. Synthesis by solid phase techniques of heterocyclic organic compounds in which N-substituted glycine monomer units forms a backbone is described in copending application entitled “Synthesis of N—Substituted Oligomers” filed on Jun. 7, 1995 and is herein incorporated by reference in full. Combinatorial libraries of mixtures of such heterocyclic organic compounds can then be assayed for the ability to alter gene expression.

Synthesis by solid phase of other heterocyclic organic compounds in combinatorial libraries is also described in copending application U.S. Ser. No. 08/485,006 entitled “Combinatorial Libraries of Substrate-Bound Cyclic Organic Compounds” filed on Jun. 7, 1995, herein incorporated by reference in full. Highly substituted cyclic structures can be synthesized on a solid support by combining the submonomer method with powerful solution phase chemistry. Cyclic compounds containing one, two, three or more fused rings are formed by the submonomer method by first synthesizing a linear backbone followed by subsequent intramolecular or intermolecular cyclization as described in the same application.

Ribozymes and Antisense

Where the therapeutic agent is a ribozyme, for example, a ribozyme targeting a portion of HIV for accomplishing a reduction of the viral load in a patient, the ribozyme can be chemically synthesized or prepared in a vector for a gene therapy protocol including preparation of DNA encoding the ribozyme sequence. The synthetic ribozymes or a vector for gene therapy delivery can be encased in liposomes for delivery, or the synthetic ribozyme can be administered with a pharmaceutically acceptable carrier. A ribozyme is a polynucleotide that has the ability to catalyze the cleavage of a polynucleotide substrate. Ribozymes for inactivating a portion of HIV can be prepared and used as described in Long et al., FASEB J. 7: 25 (1993) and Symons, Ann. Rev. Biochem. 61: 641 (1992), Perrotta et al., Biochem. 31: 16, 17 (1992); and U.S. Pat. Nos. 5,225,337, 5,168,053, 5,168,053 and 5,116,742, Ojwang et al., Proc. Natl. Acad. Sci. USA 89: 10802-10806 (1992), U.S. Pat. No. 5,254,678 and in U.S. Pat. Nos. 5,144,019, 5,225,337, 5,116,742, 5,168,053. Preparation and use of such ribozyme fragments in a hammerhead structure are described by Koizumi et al., Nucleic Acids Res. 17:7059-7071(1989). Preparation and use of ribozyme fragments in a hairpin structure are described by Chowrira and Burke, Nucleic Acids Res. 20:2835 (1992).

The hybridizing region of the ribozyme or of an antisense polynucleotide may be modified by linking the displacement arm in a linear arrangement, or alternatively, may be prepared as a branched structure as described in Horn and Urdea, Nucleic Acids Res. 17:6959-67 (1989). The basic structure of the ribozymes or antisense polynucleotides may also be chemically altered in ways quite familiar to those skilled in the art.

Chemically synthesized ribozymes and antisense molecules can be administered as synthetic oligonucleotide derivatives modified by monomeric units. Ribozymes and antisense molecules can also be placed in a vector and expressed intracellularly in a gene therapy protocol.

Protocol

Practice of the invention includes establishing that the HV-infected patient has a measurable viral load. A measurable viral load is a detectable amount of virus in the patient, detected by any means capable of detecting virus in humans. Measurement of the viral load can be accomplished by any means capable of directly or indirectly assessing virus replication by assays performed on blood cells, or tissue, serum, and plasma of the patient, as described by Voldberding and Jacobson, AIDS CLNCAL REVIEW, (Marcel Dekker, Inc. NY 1992). Viral load is variously defined in the literature and among scientists, including definitions set forth in Coombs, Clinics in Laboratory Medicine 14: 310-311(1994), providing that viral load refers to three aspects of HIV-1 replication, and to quantitative and semiquantitative assays for assessing these replication modes. The importance of the measure of a viral load in a patient is established in the art. The abundance of virus, the viral load, is recognized as an important determinant of the outcome of infection with many viruses, including HIV and other lentivirus infections. Viral load is correlated with pathogenicity, disease stage, and progression of disease, and mortality is correlated with the level of virus in the patient as described in Nowak and Bangham, Science 272:74 (1996).

Measurement of viral load in a patient can be accomplished, for example, by polymerase chain reaction (PCR) amplification against reverse transcribed HIV RNA or HIV DNA, for example as described in WO 94/20640. Alternatively, viral load can be identified by bDNA assay against RNA or DNA of HIV. bDNA can be used to detect HIV RNA or DNA, particularly to determine a viral load in a patient's plasma, cells or tissues. bDNA technology is described, for example, in U.S. Pat. No. 5,124,246, and 4,868,105. bDNA is described generally in Urdea et al NUCLEIC ACID RESEARCH SYMPOSIUM SERIES No. 24, pages 197-200 (Oxford University Press 1991). Additionally, hybridization probes can be used to detect HV DNA or RNA, using standard nucleic acid hybridization techniques. Presently, a detectable viral load for an assay against HIV RNA in plasma is about 5,000 copies of HIV RNA per mL of plasma. This detection level may change as the sensitivity of the assays for measuring viral load increases.

Elimination of HIV in an infected patient can be accomplished by a protocol that includes reducing the viral load of the patient, followed by administration of a therapeutic agent capable of increasing the CD4 T-cell count in the patient, followed or contemporaneous with an administration of a therapeutic agent capable of increasing a patient's CTLs that target HIV-infected cells. Alternatively, all these steps can be accomplished, for example, by administration of the therapeutic agents used in the protocol at the same time, rather than sequentially, for example, in the form of a therapeutic composition that includes several therapeutic agents, together accomplishing the individual tasks embodied in the therapy.

To reduce the viral load of the patient, a therapeutic agent, including a combination of therapeutic agents, including a chemotherapeutic agent, alone, or in combination with other therapeutic agents can be administered to the patient. Such agents can be for example, inhibitors of HIV enzymes, for example an inhibitor of HIV protease, an inhibitor of HIV reverse transcriptase, or an inhibitor of HIV integrase. The agent can also be an inhibitor of a biological interaction occurring in any part of the HIV life cycle, for example, an inhibitor of a tat/tar interaction or a rev/rre interaction. Chemotherapeutic agents that reduce the viral load of a patient can be, for example, a protease inhibitor, such as, for example, Sequinivir, Indinavir, Nelfinaivir, and Ritonavir, a reverse transcriptase inhibitor such as for example a non-nucleoside inhibitor or a nucleoside inhibitor including, for example lamivudine (3TC), didanosine (ddl), stavudine, lamivudine, zidovudine (AZT), zalcitabine (ddC), and delavirdine, or an integrase inhibitor, for example a small molecule inhibitor of the integrase enzyme of HIV. Any viral load reducer can additionally be used in combination with other viral load reducers to achieve an optimal reduction in viral load in the patient. Thus, for example, a protease inhibitor can be used in combination with a reverse transcriptase inhibitor, or with more than one reverse transcriptase inhibitor, such as described in Carpenter et al, JAMA 276: 146-154 (1996). Similarly, for example, an integrase inhibitor can be used in combination with a reverse transcriptase inhibitor, or a protease inhibitor, or both. Further, an inhibitor of some other biological interaction in the HIV life cycle, such as a tat/tar interaction or a rev/rre interaction, can be used in combination with an integrase inhibitor, a protease inhibitor, or a reverse transcriptase inhibitor, or a combination of those inhibitors. An example of a favorable combination of chemotherapeutic agents including administering a combination of zidovudine with lamivudine and Indivinavir.

Any agent that inhibits the action of an HIV protease, an HIV reverse transcriptase, an HIV integrase, or that inhibits a biological interaction involved in the HIV life cycle, can be an effective viral load reducer, including, for example, a polynucleotide, a polypeptide, an organic small molecule, a peptide, or a peptoid inhibitor.

After the viral load in the patient has been reduced, or simultaneous with reduction of the viral load in the patient, the invention provides for increasing the amount of CD4 T-cells in the patient by administration of, for example, a T-cell growth factor, or a cytokine known to induce endogenous production of CD4 T-cells in patients. Increasing the CD4 T-cell count in a patient is accompanied by a return of a delayed hypersensitivity cellular immune response to the patient, although the invention is not limited to any theories or mechanisms. Patients infected with HIV show a reduced or absent delayed-type hypersensitive immune response, which is an important host defense mechanism against intracellular pathogens, as described in Kuby, IMMUNOLOGY, (W.H. Freeman & Co., NY 1992) pp. 475-477. Administration of a therapeutic agent that increases the number of healthy CD4 T-cells in the patient is accompanied by a return of normal CD4 T-cell function, including, for example the return of a delayed type hypersensitivity that is mediated by sensitized T-lymphocytes. The response is characterized by the release of growth and differentiation factors in response to foreign antigen with the recruitment and activation of macrophages, and the response can provide the mechanism against intracellular pathogens, described earlier, as described in Kuby, IMMUNOLOGY, (W.H. Freeman & Co., N 1992) pp. 535.

Increase of CD4 T-cells can be accomplished, for example, by administration of an agent capable of inducing or increasing the patient's endogenous production of CD4+ T-cells. This can be accomplished, for example by administering a T-cell growth factor, or a cytokine. The cytokine can be, for example, an IL-2, IL-4, IL-7, IL-9, IL-12, or gamma interferon (INFγ). The cytokine or other T-cell growth factor can be administered as a polypeptide, or as a polynucleotide in a gene therapy protocol, for expression of the cytokine in the patient. Alternatively, an inducer of a cytokine or a T-cell growth factor can be administered, for example by gene therapy or as a polypeptide agent, for inducing production of the T-cell growth factor or cytokine in the patient.

Where IL-2 is used to induce CD4 T-cell production in the patient, the IL-2 can be, for example, biologically active mature IL-2, truncated IL-2, or an IL-2 variant, such as, for example, IL-2 des Ala Ser-125. Such a protocol for induction of CD4 T-cells in a patient is described in WO 94/26293. Multiple continuous infusions of IL-2 can be administered intermittently over an extended period of time. The dosages can be in a range from 1 million international units per day to 24 million international units per day. Lower doses can also be used, depending on the dose required for effectiveness in the patient. For example, IL-2 can be administered by continuous IV infusion over 5 days, once every 8 weeks, at doses between about 6 to about 18 million international units per day. The period of time between successive infusions can vary from 4 weeks to six months, and even a year. The intermittent administration of IL-2 can be analogous to the in vitro approach of alternating cycles of stimulation with rest needed for establishment or expression of T-cell lines or clones, as described in Kimoto and Fathman, J. Exp. Med. 152: 759-70 (1980). Further, anti-retroviral therapy can commence before the IL-2 therapy is started, and can continue through the course of a intermittent IL-2 therapy.

According to one particular regime for treatment, IL-2, preferably aldesleukin, can be administered subcutaneously at a dose of 7.5 MIU every 12 hours (q12h) on days 1-5 as tolerated of an approximately 8-week cycle for a total of six cycles.

Additionally, the patients will also receive standard of care antiretroviral therapy as well as a CTL-inducing vaccine. Preferably, patients are treated with the best antiretroviral agent or a combination of antiretroviral agents for a minimum of two weeks prior to IL-2 treatment. Each cycle of subcutaneous IL-2 therapy can be administered approximately every 8 weeks. Optimally, patients can receive cycle 2 and/or all subsequent cycles as early as week 7 of a given cycle or as late as week 9. A given cycle may be extended to as late as week 11 in exceptional circumstances, but the overall duration of an individual's protocol participation should not extend beyond 15 months.

Alternatively, the IL-2 can be administered by a gene therapy protocol, that takes advantage of the activated state of the immune system during the course of the IL-2 treatment. T-cells can be obtained from the patient, transduced in vitro, and infused into the patient. Perhaps to better effect, the immune system can be activated by administering IL-2, for example, in the intermittent administration protocol just described, and the IL-2 induces the cells to become activated and to synthesize DNA which makes them more receptive to transduction by a viral vector, for example a retroviral vector, a non-viral vector, or naked DNA. A genetically engineered retroviral vector, for example, can be administered directly to the patient, and this vector, once integrated in the patient's DNA can express the gene in the vector. The gene in the vector could be, for example, IL-2, an inducer of IL-2 production, or other gene useful for a treatment of an HIV-infected patient. The vector could also contain, for example a non-coding sequence, for example an antisense polynucleotide, or a ribozyme, capable of targeting an HIV nucleic acid sequence, for further arresting the viral life cycle, or for acting in prophylaxis of further infection of the transformed T-cell.

Where administration of IL-2 or other cytokine or T-cell growth factor is conducted in a gene therapy protocol, the therapeutic agent for increasing a CD4 T-cell count can be administered as naked DNA, with a non-viral vector, or with a viral vector, for example a retroviral vector, using methods as described, for example, below. Additionally, a therapeutic agent can be administered that induces endogenous expression of the cytokine capable of increasing the production of CD4 T-cells in the patient, such as, for example, an agent capable of inducing endogenous production of IL-2 in the patient. Such a therapeutic agent capable of inducing an endogenous T-cell growth factor, that then induces in vivo CD4 T-cells, can be administered as a potypeptide therapeutic, a small molecule, such as an organic small molecule or a peptoid, a peptide, or a polynucleotide. The polynucleotide can be administered in a gene therapy protocol for administering a polynucleotide therapeutic agent that is then expressed in the patient to achieve the desired effects.

One particular virus vector for introduction of one or more of the therapeutic agents of the present invention is based on Sindbis virus. This vector called ELVS™ exploits the amplification properties of Sindbis virus in conjunction with normal plasmid DNA delivery. Briefly, the vector consists of a nucleic acid vector containing its own replicase (NSP) which in turns recognizes a viral cis acting sequence (JR) resulting in transcription and amplification of the desired gene of interest (GOI). Theoretically, this vector system has an inherent advantage in that very small amounts of DNA are necessary for expression and immunization. In one useful vector system Sindbis virus-derived sequences including four nonstructural protein genes, complete 5′- and 3′-end untranslated regions, subgenomic promoter (JR), and polyA tract (A₄₀) are used, for example, with the cytomegalovirus immediate early promoter (CMV), hepatitis delta virus antigenomic ribozyme sequence (δ) bovine growth hormone transcription terminations signal (TT). Design of suitable vectors is well within the skill of the art.

The gp120, gp160/rev, and gagpol/rev genes from B and E dade HIV viruses can be expressed in conventional CMV plasmids as well as in the ELVS™ vector. In all cases where RRE (Rev-response element) sequences or CRS (cis-acting repressor element) sequences are present in the HIV DNA sequences it is desirable to have Rev co-expression using differential spliced expression of the rev exons.

One particular CMV vector, CMVKm2, utilizes the human CMV immediate early promoter/intron A and the bGH termination signals. HIV Env signal sequences can be replaced by the tPA leader to enhance protein secretion. Env expression can be confirmed by in vitro transfection of various cell lines followed by immunoblotting; expression levels can be determined in transfected cell supernatants by antigen capture ELISA. ELVS™ vector also utilizes the human CMV immediate early promoter/intron A and the bGH termination signals except that an amplification system is added to the expression system. See Chapman, NAR 19: 3979, 1991. Pox virus vectors, retroviral virus vectors, AAV vectors and alphavirus vectors may also be used.

Once the viral load has been reduced and the CD4 T-cell population has been increased, or simultaneous with an increase in CD4 T-cells, or simultaneous with a reduction of viral load and increase of CD4 T-cells, the patient's CTLs targeting HIV-infected cells are increased. The CTLs targeted to HIV-infected cells detect and eliminate the HIV-infected cells from the patient, although the invention is not limited by any theories or mechanisms.

The patient's HIV-targeted CTLs can be increased by administering a vaccine to the patient. It is acknowledged that other therapeutic methods for increasing CTLs in the patient may exist, and as such these methods can be used to achieve an increase of CTLs targeting HIV-infected cells in the patient, and as such are contemplated to be within the scope of usefulness for achieving the invention. Where a vaccine is administered to a patient to accomplish an increase in the HIV specific CTLs in the patient, it is also acknowledged that administration of a vaccine to the patient, in addition to increasing the CTLs in the patient that target HIV-infected cells, can have other effects on the immune system which may be beneficial in promoting the ultimate recovery of the patient. For example, in addition to increasing the CTLs in the patient, an anti-HIV vaccine may improve helper T-cell function, and may also provide epitopes that induce neutralizing antibodies in the patient that target HIV antigens. The vaccine to be administered is particulary designed to induce the patient's production of CTLs specific for HIV-infected cells, but it is acknowledged that in addition to the CTL enhancement of numbers and function, other beneficial immunologically-based effects may occur in the patient and may contribute to the improved health of the patient.

The vaccine for inducing CTLs in the patient that target HIV-infected cells is designed based on the HIV genome and viral structure. The vaccine can be a subunit based vaccine or a nucleic acid vaccine, both based on the identity of HIV genes. A subunit vaccine will include a polypeptide subunit of the HIV genome, for example with an adjuvant, matrix, or pharmaceutically acceptable carrier. A nucleic acid vaccine is also based on HIV genes, but provides a gene encoding all or a part of, or a fusion, chimera, or altered variant of, an HIV polypeptide. The nucleic acid vaccine is delivered in a vaccination protocol, for example, in a protocol including a pharmaceutically acceptable carrier. The advantage provided by a nucleic acid vaccine, including a DNA or RNA-based vaccine, is that expression of the molecule that stimulates production of CTLs targeted to HIV-infected cells occurs in vivo, in the patient's cells, and can result in an expression product most likely to activate the CTLs to the endogenous HIV-infected cells. For example, proper glycosylation or post-translation modification will occur during the protein expression. Induction of CTL responses can be achieved using DNA inoculation of patients. For example, inoculation with a gp160 DNA construct which encodes HIV gp160 followed by boosting caused specific cross-reactive cytotoxic T lymphocyte responses in vaccinated primates. Wang et al., Virology 211:102-112 (1995). Similar inoculations using env protein and the Rev regulatory protein of HIV in mice or macaques induced a strong cytotoxic T lymphocyte (CTL) response against target cells pulsed with the V3 peptide. Okuda et al., AIDS Research and Human Retroviruses, 11:933-945 (1995). Thus, DNA inoculations, like protein inoculations have been demonstrated to achieve specific CTL responses.

Subunit-based polypeptides are chosen to be capable of effectively activating CTLs that target HV-infected cells. The selected subunit or polyprotein, or fusion protein can be cloned and expressed in a recombinant system, for example, a bacterial, yeast, insect, amphibian, or mammalian system. The HIV genome including, for example, the gag, pol, env, tat, and rev genes, can form the basis of selection and design of the subunit vaccine. Other genes, known as the accessory genes including vif, vpr, vpu and particularly including nef, may be useful in constructing an effective subunit vaccine as well. A thorough description of structure and function of the HIV genes is provided in Fields et al, VIROLOGY (3rd Ed. Lippincott-Raven, Phil, Pa. 1996) vol 2, ch. 60, pp. 1881-1952. The gag gene, for example, generates the polyprotein Pr55 gag, and the polypeptide p24, which can form the basis of a polypeptide based vaccine for increasing a patient's CTLs targeting HIV-infected cells. Likewise the pol gene yields the polyprotein precursor Pr160 gag-pol, which is a precursor for virion enzymes HIV protease (PR) or p10, HIV reverse transcriptase (RT and RNAse-H) or p51/66, and integrase (IN) p32, and, for example, these polyproteins or subunits can be used to generate a vaccine. Additionally, the env gene yields the precursor for envelope glycoprotein gp160 and its components called SU or gp120, and TM or gp41, which can form the basis of a subunit vaccine. HIV derived polypeptide components are described in EP 201 540, EP 181 150 B1, and U.S. Pat. No. 4,725,669, both incorporated by reference in full. The gp160 polyprotein, or gp120, or gp41 subunits can be used individually to generate a vaccine, or can be used together, for example in a fusion protein including for example, all of gp120 and a portion of gp41 in a fusion protein. Other polyproteins precursors and polypeptide subunits of HIV may also form the basis of a subunit vaccine, including, for example any HIV gene or portion of an HIV gene capable of being recombinantly expressed and delivered in a vaccination protocol. Any of the polyproteins or subunits can be fused in a fusion protein or chimera for generation of a CTL population most effective in targeting HIV-infected T-cells. The most effective subunit or subunit-based polypeptide fusion for development of a vaccine to increase specific CTL production in the patient will be that subunit that, when delivered in a vaccine, induces a CTL response in the patient that is effective and specific for the patient's HIV-infected cells. The subunits used in development of the vaccine can be all or part of any HIV subunit or polyprotein precursor. Fusion proteins can include, for example, fusions of gal and pol subunits of an HIV gene, or a fusion protein gp140 having a fusion of gp120 and at least a portion of gp41 subunits of an HIV gene. The subunit vaccine can also be made of an immunogenic molecule such as a peptide derivative of an HIV subunit, or an epitope derived from an HIV gene, provided the immunogenic molecule comprises a molecule capable of an immune response in the patient including induction of CTLs in the patient. In all cases, the vaccine based on an HIV subunit or polyprotein precursor can also or separately produce an induction of lymphocytes with T-cell helper function, or an induction of antibodies capable of nuetralizing HIV.

For example, the p55 gag protein, particularly the p24 subunit of this protein, can be a component of a vaccine for targeting CD8+ CTL responses in HIV infected patients, where the vaccine is used to prime virus-specific cytotoxic cells against this highly conserved viral protein. The vaccination using p55 gag protein can be used to accomplish priming of class 1 MHC—(major histocompatibility complex)—restricted CD8 CTL responses, which priming usually requires expression of proteins in the cytosol or endoplasmic reticulum of antigen-presenting cells (APCs). This priming effect can be achieved by administration of recombinant viral or plasmid DNA vaccines. It is also believed that the recombinant viral proteins can enter the class I MHC processing pathway when formulated with specialized adjuvants, for example, model proteins formulated with carrier beads as described in Kovacsovics-Bankowski et al, PNAS USA 90: 49424946 (1993), liposomes, cationic lipids, and oil inwater emulsion adjuvants.

A nucleic acid vaccine can be an RNA, a DNA or a synthetic polynucleotide vaccine. Administration of DNA and mRNA vaccines are described, for example, in WO 90/11092, incorporated by reference in full. Nucleic acid vaccines are distinguished from a simple gene therapy protocol, although related to gene therapy, in that the nucleic acids are delivered in a vaccination protocol that is designed to elicit a therapeutic immune response in the patient. Gene therapy delivery of nucleic acids is provided for the introduction of genes into a patient for expression of the gene in the patient, the expressed gene product not necessarily eliciting an immune response in the patient, but perhaps achieving other effects facilitated by activity of the expressed gene product.

A nucleic acid immunization is the introduction of a nucleic acid molecule encoding one or more selected antigens into a host cell, for the in vivo expression of the antigen or antigens. The nucleic acid molecule can be introduced into a patient, for example, by injection, particle gun, topical administration, parental administration, inhalation, or iontophoretic delivery, as described in U.S. Pat. Nos. 4,411,648 and 5,222,936, 5,286,254; and WO 94/05369. More description of exemplary administrations and delivery for vaccines is provided below. Any polynucleotide coding sequence encoding an antigen which is a candidate for inducing production of CTLs in a patient that can target HIV-infected cells can be used with success in a nucleic acid vaccine for this invention. Additionally, the vaccination may generate an immune response, including a humoral or cellular immune response, for example an antibody response or an augmentation of helper T-cell function, in addition to the CTL HIV-infected cell targeting response.

Polynucleotide sequences coding for the a molecules capable of inducing the endogenous production of CTLs in a patient can be obtained using recombinant methods, such as by screening cDNA and genomic libraries from cells expressing the gene, or by deriving the gene from a vector that carries the gene. The desired gene can also be isolated from cells and tissues containing the gene, using phenol extraction, PCR of cDNA, or genomic DNA. The gene of interest can also be produced synthetically, rather than cloned, as described in Edge, Nature 292: 756 (1981), Nambair et al, Science 223: 1299 (1984), and Jay et al, J. Biol. Chem. 259: 6311 (1984).

The nucleic acid vaccine can include all or a part of the HIV genome. In addition, the nucleic acid vaccine can include a polynucleotide sequence encoding a fusion protein or chimera of two or more HIV subunits or polyproteins. The HIV genome including, for example, the gag, pol, env, tat, and rev genes, can form the basis of selection and design of the nucleic acid vaccine. Other genes, known as the accessory genes including vif, vpr, vpu and particularly including nef, may be useful in constructing an effective subunit vaccine as well. A thorough description of structure and function of the HV genes is provided in Fields et al, VIROLOGY (3rd Ed. Lippincott-Raven, Phil, Pa. 1996) vol 2, ch. 60, pp. 1881-1952. The gene gag, for example, generates the polyprotein Pr55 gag, and the polypeptide p24, which can form the basis of a polynucleotide based vaccine for increasing a patient's CTLs targeting HIV-infected cells. Likewise the pol gene yields the polyprotein precursor Pr160 gag-pol, which is a precursor for virion enzymes HIV protease (PR) or p10, HIV reverse transcriptase (RT and RNAse-H) or p51/66, and integrase (IN) p32, and, for example, the polynucleotide sequences encoding these polyproteins or subunits can be used to generate a nucleic acid vaccine. Additionally, the env gene yields the precursor for envelope glycoprotein gp160 and its components called SU or gp120, and TM or gp41, which can form the basis of a nucleic acid vaccine. The gp120 protein is described in WO 91/13906 and HIV-1 envelope protein muteins based on gp120 are described in EP 434 713. A polynucleotide encoding the gp160 polyprotein, or gp120, or gp41 subunits can be used individually to generate a vaccine, or can be used together, for example in a polynucleotide encoding a fusion protein including for example, all of gp120 and a portion of gp41 in a fusion protein. Other polyproteins precursors and polypeptide subunits of HIV may also form the basis of a nucleic acid vaccine, including, for example any HIV gene or portion of an HIV gene capable of being recombinantly expressed and delivered in a vaccination protocol. Additionally, noncoding regions of the HIV genome may be used to effect in a nucleic acid vaccine, for example, to control expression of the antigenic polypeptide. Additionally, a polynucleotide encoding any of the polyproteins or subunits in a fused coding sequence can be used to generate a CTL population in the patient that is most effective for targeting HIV-infected T-cells. The most effective polynucleotide encoding a subunit or subunit-based polypeptide fusion for development of a vaccine to increase specific CTL production in the patient will be that polynucleotide that encodes a subunit or fusion that, when delivered in a vaccine, induces a CTL response in the patient that is effective and specific for the patient's HIV-infected cells. The subunits used in development of the nucleic acid vaccine can be all or part of any HV subunit or polyprotein precursor. Fusion genes encoding fusion proteins can include, for example, fusions of gal and pol subunits of an WV gene, or a fusion protein gp140 having a fusion of gp120 and at least a portion of gp41 subunits of an HIV gene. The nucleic acid vaccine can also be made of a polynucleotide encoding an immunogenic molecule such as a peptide derivative of an HIV subunit, or an epitope derived from an HIV gene, provided the immunogenic molecule comprises a molecule capable of an immune response in the patient including induction of CTLs in the patient. In all cases, the nucleic acid vaccine based on an HIV subunit or polyprotein precursor may also induce lymphocytes with T-cell helper function, or induce antibodies capable of nuetralizing HIV. However, only induction of CTLs targeting HIV-infected cells is required for this prong of the invention.

The vaccine will contain an antigen, or a polynucleotide encoding an antigen, usually in combination with pharmaceutically acceptable carriers, which include any carrier that does not itself induce the production of antibodies harmful to the individual receiving the composition. Suitable carriers for a vaccine are typically large, slowly metabolized macromolecules such as proteins, polysaccharides, polylactic acids, polyglycolic acids, polymeric amino acids, amino acid copolymers, lipid aggregates (such as oil droplets or liposomes), and inactive virus particles. Such carriers are well known to those of ordinary skill in the art. Additionally, these carriers may function as immunostimulating agents also called adjuvants. Furthermore, the antigen may be conjugated to a bacterial toxoid, such as a toxoid from diphtheria, tetanus, cholera, H. pylori, etc.

Preferred adjuvants to enhance effectiveness of the composition include, but are not limited to: (1) aluminum salts (alum), such as aluminum hydroxide, aluminum phosphate, aluminum sulfate, etc; (2) oil-in-water emulsion formulations (with or without other specific immunostimulating agents such as muramyl peptides (see below) or bacterial cell wall components, such as for example (a) MF59 (PCT Publ. No. WO 90/14837), containing 5% Squalene, 0.5% Tween 80, and 0.5% Span 85 (optionally containing various amounts of MTP PE (see below), although not required) formulated into submicron particles using a microfluidizer such as Model 110Y microfluidizer (Microfluidics, Newton, Mass.), (b) SAF, containing 10% Squalane, 0.4% Tween 80, 5% pluronic-blocked polymer L121, and thr-MDP (see below) either microfluidized into a submicron emulsion or vortexed to generate a larger particle size emulsion, and (c) RibiTM adjuvant system (RAS), (Ribi Immunochem, Hamilton, Mont.) containing 2% Squalene, 0.2% Tween 80, and one or more bacterial cell wall components from the group consisting of monophosphorylipid A (MPL), trehalose dimycolate (TDM), and cell wall skeleton (CWS), preferably MPL+CWS (DetoxTM); (3) saponin adjuvants, such as StimulonTM (Cambridge Bioscience, Worcester, Mass.) may be used or particles generated therefrom such as ISCOMs (immunostimulating complexes); (4) Complete Freunds Adjuvant (CFA) and Incomplete Freunds Adjuvant (IFA); (5) cytokines, such as interleukins (e.g., IL-1, IL2, IL-4, IL-5, IL-6, IL-7, IL-12, etc.), interferons (e.g., gamma interferon), macrophage colony stimulating factor (M-CSF), tumor necrosis factor (TNF), etc; and (6) other substances that act as immunostimulating agents to enhance the effectiveness of the composition. Alum and MF59 are preferred.

As mentioned above, muramyl peptides include, but are not limited to, Nacetyl-muramyl-L-threonyl-D-isoglutamine (thr-MDP), N-acetyl-normuramyl-L-alanyl-D-isoglutamine (nor-MDP), N-acetylmuramyl-L-alanyl-D-isoglutaminyl-L-alanine-2-(1′-2′-dipalmitoy 1-sn-glycero-3-huydroxyphosphoryloxy)-ethylamine (MTP-PE), etc.

The immunogenic compositions (e.g., the antigen, pharmaceutically acceptable carrier, and adjuvant) typically will contain diluents, such as water, saline, glycerol, ethanol, etc. Additionally, auxiliary substances, such as wetting or emulsifying agents, pH buffering substances, and the like, may be present in such vehicles.

Typically, the immunogenic compositions are prepared as injectables, either as liquid solutions or suspensions; solid forms suitable for solution in, or suspension in, liquid vehicles prior to injection may also be prepared. The preparation also may be emulsified or encapsulated in liposomes for enhanced adjuvant effect, as discussed above under pharmaceutically acceptable carriers. Immunogenic compositions used as vaccines comprise an immunologically effective amount of the antigenic polypeptides, as well as any other of the above-mentioned components, as needed. By the term immunologically effective amount, it is meant that the administration of that amount to an individual, either in a single dose or as part of a series, that is effective for treatment or prevention. This amount varies depending upon the health and physical condition of the individual to be treated, the taxonomic group of individual to be treated (for example whether the patient is a human, ., nonhuman primate, or primate), the capacity of the individual's immune system to synthesize antibodies, the degree of protection desired, the formulation of the vaccine, the treating doctor's assessment of the medical situation, and other relevant factors. It is expected that the amount will fall in a relatively broad range that can be determined through routine trials.

The immunogenic compositions are conventionally administered parenterally, for example by injection, either subcutaneously or intramuscularly. Additional formulations suitable for other modes of administration include oral and pulmonary formulations, suppositories, and transdermal applications. Dosage treatment may be a single dose schedule or a multiple dose schedule. The vaccine may be administered in conjunction with other immunoregulatory agents.

Gene therapy strategies for delivery of constructs including a coding sequence of a therapeutic of the invention, to be delivered to the patient for expression in the patient, for example, an IL-2 coding sequence, or also including a nucleic acid sequence of all or a portion of the HIV genome for delivery in a vaccination protocol for generation of an immune response, including CTL induction, can be administered by a gene therapy protocol, either locally or systemically. These construct can utilize viral or non-viral vector approaches in in vivo or ex vivo modality. Expression of such coding sequence can be induced using endogenous mammalian or heterologous promoters. Expression of the coding sequence in vivo can be either constitutive or regulated.

Administration of a nucleic acid vaccine, or a gene for expression in the patient in a gene therapy protocol, can be provided by a viral vector, including for example, a vector of a retrovirus, an adenovirus, an adeno-associated virus, a herpes virus, a sindbis virus, including sindbis DNA or sindbis RNA, or ELVS DNA. Further examples of viral vectors are described in Jolly, Cancer Gene Therapy 1: 51-64 (1994). The coding sequence of a desired polypeptide or ribozymes or antisense molecules can also be inserted into plasmids designed for transcription and/or translation in retroviral vectors, as described in Kimura et al., Human Gene Therapy (1994) 5: 845-852, adenoviral vectors, as described in Connelly et al., Human Gene Therapy (1995) 6: 185-193, adeno-associated viral vectors, as described in Kaplitt et al., Nature Genetics (1994) 6: 148-153 and sindbis vectors. Promoters that are suitable for use with these vectors include the Moloney retroviral LTR, CMV promoter and the mouse albumin promoter. Replication incompetent free virus can be produced and injected directly into the animal or humans or by transduction of an autologous cell ex vivo, followed by injection in vivo as described in Zatloukal et al, Proc. Natl. Acad Sci. USA (1994) 91: 5148-5152.

The polynucleotide encoding a desired polypeptide or ribozyme or antisense polynucleotide can also be inserted into plasmid for delivery to cells and where the polynucleotide is a coding sequence, for expression of the desired polypeptide in vivo. Promoters suitable for use in this manner include endogenous and heterologous promoters such as CMV. Further, a synthetic T7T7/T7 promoter can be constructed in accordance with Chen et al. (1994), Nucleic Acids Res. 22: 2114-2120, where the T7 polymerase is under the regulatory control of its own promoter and drives the transcription of polynucleotide sequence, which is also placed under the control of a T7 promoter. The polynucleotide can be injected in a formulation that can stablize the coding sequence and facilitate transduction thereof into cells and/or provide targeting, as described in Zhu et al., Science (1993) 261: 209-211.

Expression of the coding sequence of a desired polypeptide or replication of a ribozyme or antisense polynucleotide in vivo upon delivery for gene therapy purposes by either viral or non-viral vectors can be regulated for maximal efficacy and safety by use of regulated gene expression promoters as described in Gossen et al., Proc. Natl. Acad Sci. USA (1992) 89:5547-5551. For example, the polynucleotide transcription and/or translation can be regulated by tetracycline responsive promoters. These promoters can be regulated in a positive or negative fashion by treatment with the regulator molecule.

For non-viral delivery of the coding sequence, the sequence can be inserted into conventional vectors that contain conventional control sequences for high level expression, and then be incubated with synthetic gene transfer molecules such as polymeric DNA-binding cations like polylysine, protamine, and albumin, linked to cell targeting ligands such as asialoorosomucoid, as described in Wu and Wu, J. Biol Chem. (1987) 262: 4429-4432; insulin, as described in Hucked et al., Biochem. Pharmacol. 40: 253-263 (1990); galactose, as described in Plank et al., Bioconjugate Chem. 3:533-539 (1992); lactose, as described in Midoux et al., Nucleic Acids Res. 21: 871-878 (1993); or transferrin, as described in Wagner et al., Proc. Natl. Acad. Sci. USA 87:3410-3414 (1990). Other delivery systems include the use of liposomes to encapsulate DNA comprising the gene under the control of a variety of tissue-specific or ubiquitously-active promoters, as described in Nabel et al., Proc. Natl. Acad. Sci. USA 90: 11307-11311 (1993), and Philip et al., Mol. Cell Biol. 14: 2411-2418 (1994). Further non-viral delivery suitable for use includes mechanical delivery systems such as the biolistic approach, as described in Woffendin et al, Proc. Natl. Acad. Sci. USA (1994) 91(24): 11581-11585. Moreover, the coding sequence and the product of expression of such can be delivered through deposition of photopolymerized hydrogel materials. Other conventional methods for gene delivery that can be used for delivery of the coding sequence include, for example, use of hand held gene transfer particle gun, as described in U.S. Pat. No. 5,149,655; use of ionizing radiation for activating transferred gene, as described in U.S. Pat. No. 5,206,152 and PCT application WO 92/11033. The aforementioned are not to the exclusion of additional means of facilitating of nucleic acid uptake that rely on nucleic charge neutralization or fusion with cell membranes or facilitate uptake, for example.

Administration of a nucleic acid vaccine or a gene for expression in the patient for a non-immunological effect, or a non-coding polynucleotide sequence, can be accomplished by use of a polypeptide, a peptide, a conjugate, a liposome, a lipid, a viral vector, for example, a retroviral vector a non-viral vector.

Polycationic molecules, lipids, liposomes, polyanionic molecules, or polymer conjugates conjugated to the polynucleotide can facilitate non-viral delivery of DNA or RNA. For example, polycationic agents for gene delivery include: polylysine, polyarginine, polyornithine, and protamine. Other examples include histones, protamines, human serum albumin, DNA binding proteins, non-histone chromosomal proteins, coat proteins from DNA viruses, such as φX174, transcriptional factors also contain domains that bind DNA and therefore may be useful as nucleic aid condensing agents, for example, C/CEBP, c-jun, c-fos, AP-1, AP-2, AP-3, CPF, Prot-1, Sp-1, Oct-1, Oct-2, CREP, and TFIID contain basic domains that bind DNA sequences. Organic polycationic agents include: spermine, spermidine, and purtrescine. The dimensions and of the physical properties of a polycationic agent can be extrapolated from the list above, to construct other polypeptide polycationic agents or to produce synthetic polycationic agents.

Gene delivery vehicles (GDVs) are available for delivery of polynucleotides to cells, tissue, or to a the mammal for expression. For example, a polynucleotide sequence of the invention can be administered either locally or systemically in a GDV. These constructs can utilize viral or non-viral vector approaches in in vivo or ex vivo modality. Expression of such coding sequence can be induced using endogenous mammalian or heterologous promoters. Expression of the coding sequence in vivo can be either constitutive or regulated. The invention includes gene delivery vehicles capable of expressing the contemplated polynucleotides. The gene delivery vehicle is preferably a viral vector and, more preferably, a retroviral, adenoviral, adeno-associated viral (AAV), herpes viral, or alphavirus vectors. The viral vector can also be an astrovirus, coronavirus, orthomyxovirus, papovavirus, paramyxovirus, parvovirus, picornavirus, poxyirus, togavirus viral vector. See generally, Jolly, Cancer Gene Therapy 1:51-64 (1994); Kimura, Human Gene Therapy 5:845-852 (1994), Connelly, Human Gene Therapy 6:185-193 (1995), and Kaplitt, Nature Genetics 6:148-153 (1994).

Retroviral vectors are well known in the art and we contemplate that any retroviral gene therapy vector is employable in the invention, including B, C and D type retroviruses, xenotropic retroviruses (for example, NZB-X1, NZB-X2 and NZB9-1 (see O'Neill, J. Vir. 53:160, 1985) polytropic retroviruses (for example, MCF and MCF-MLV (see Kelly, J. Vir. 45:291, 1983), spumaviruses and lentiviruses. See RNA Tumor Viruses, Second Edition, Cold Spring Harbor Laboratory, 1985. Portions of the retroviral gene therapy vector may be derived from different retroviruses. For example, retrovector LTRs may be derived from a Murine Sarcoma Virus, a tRNA binding site from a Rous Sarcoma Virus, a packaging signal from a Murine Leukemia Virus, and an origin of second strand synthesis from an Avian Leukosis Virus. These recombinant retroviral vectors may be used to generate transduction competent retroviral vector particles by introducing them into appropriate packaging cell lines (see U.S. Ser. No. 07/800,921, filed Nov. 29, 1991). Retrovirus vectors can be constructed for site-specific integration into host cell DNA by incorporation of a chimeric integrase enzyme into the retroviral particle. See, U.S. Ser. No. 08/445,466 filed May 22, 1995.

It is preferable that the recombinant viral vector is a replication defective recombinant virus. Packaging cell lines suitable for use with the above-described retrovirus vectors are well known in the art, are readily prepared (see U.S. Ser. No. 08/240,030, filed May 9, 1994; see also WO 92/05266), and can be used to create producer cell lines (also termed vector cell lines or “VCLs”) for the production of recombinant vector particles. Preferably, the packaging cell lines are made from human parent cells (e.g., Hr1080 cells) or mink parent cell lines, which eliminates inactivation in human serum. Preferred retroviruses for the construction of retroviral gene therapy vectors include Avian Leukosis Virus, Bovine Leukemia, Virus, Murine Leukemia Virus, Mink-Cell Focus-Inducing Virus, Murine Sarcoma Virus, Reticuloendotheliosis Virus and Rous Sarcoma Virus. Particularly preferred Murine Leukemia Viruses include 4070A and 1504A (Hartley and Rowe, J. Virol. 19:19-25, 1976), Abelson (ATCC No. VR-999), Friend (ATCC No. VR-245), Graffi, Gross (ATCC No. VR-590), Kirsten, Harvey Sarcoma Virus and Rauscher (ATCC No. VR-998) and Moloney Murine Leukemia Virus (ATCC No. VR-190). Such retroviruses may be obtained from depositories or collections such as the American Type Culture Collection (“ATCC”) in Rockville, Md. or isolated from known sources using commonly available techniques.

Exemplary known retroviral gene therapy vectors employable in this invention include those described in GB 2200651; EP No. 415,731; EP No. 345,242; PCT Publication Nos. WO 89/02468, WO 89/05349, WO 89/09271, WO 90/02806, WO 90/07936, WO 90/07936, WO 94/03622, WO 93/25698, WO 93/25234, WO 93/11230, WO 93/10218, and WO 91/02805, in U.S. Pat. Nos. 5,219,740, 4,405,712, 4,861,719, 4,980,289 and 4,777,127, in U.S. Ser. No. 07/800,921 and in Vile, Cancer Res. 53:3860-3864 (1993); Vile, Cancer Res 53:962-967 (1993); Ram, Cancer Res 53:83-88 (1993); Takamiya, J. Neurosci. Res. 33:493-503 (1992); Baba, J Neurosurg 79:729-735 (1993); Mann, Cell 33:153 (1983); Cane, Proc Natl Acad Sci 81:6349(1984) and Miller, Human Gene Therapy 1 (1990).

Human adenoviral gene therapy vectors are also known in the art and employable in this invention. See, for example, Berkner, Biotechniques 6:616 (1988), and Rosenfeld, Science 252:431(1991), and PCT Patent Publication Nos. WO 93/07283, WO 93/06223, and WO 93/07282, as well as U.S. Ser. No. 08/869,309. Exemplary known adenoviral gene therapy vectors employable in this invention include those described in the above-referenced documents and in PCT Patent Publication Nos. WO 94/12649, WO 93/03769, WO 93/19191, WO 94/28938, WO 95/11984, WO 95/00655, WO 95/27071, WO 95/29993, WO 95/34671, WO 96/05320, WO 94/08026, WO 94/11506, WO 93/06223, WO 94/24299, WO 95/14102, WO 95/24297, WO 95/02697, WO 94/28152, WO 94/24299, WO 95/09241, WO 95/25807, WO 95/05835, WO 94/18922 and WO 95/09654. Alternatively, administration of DNA linked to killed adenovirus as described in Curiel, Hum. Gene Ther. 3:147-154 (1992) may be employed. The gene delivery vehicles of the invention also include adenovirus associated virus (AAV) vectors. Leading and preferred examples of such vectors for use in this invention are the AAV-2 basal vectors disclosed in Srivastava, PCT Patent Publication No. WO 93/09239. Most preferred AAV vectors comprise the two AAV inverted terminal repeats in which the native D-sequences are modified by substitution of nucleotides, such that at least 5 native nucleotides and up to 18 native nucleotides, preferably at least 10 native nucleotides up to 18 native nucleotides, most preferably 10 native nucleotides are retained and the remaining nucleotides of the D-sequence are deleted or replaced with non-native nucleotides. The native D-sequences of the AAV inverted terminal repeats are sequences of 20 consecutive nucleotides in each AAV inverted terminal repeat (i.e., there is one sequence at each end) which are not involved in HP formation. The non-native replacement nucleotide may be any nucleotide other than the nucleotide found in the native D-sequence in the same position. Other employable exemplary AAV vectors are pWP-19, pWN-1, both of which are disclosed in Nahreini, Gene 124:257-262 (1993). Another example of such an AAV vector is psub201. See Samulski, J. Virol. 61:3096 (1987). Another exemplary AAV vector is the Double-D ITR vector. How to make the Double D ITR vector is disclosed in U.S. Pat. No. 5,478,745. Still other vectors are those disclosed in Carter, U.S. Pat. No. 4,797,368 and Muzyczka, U.S. Pat. No. 5,139,941, Chartejee, U.S. Pat. No. 5,474,935, and Kotin, PCT Patent Publication No. WO 94/288157. Yet a further example of an AAV vector employable in this invention is SSV9AFABTKneo, which contains the AFP enhance and albumin promoter and directs expression predominantly in the liver. Its structure and how to make it are disclosed in Su, Human Gene Therapy 7:463-470 (1996). Additional AAV gene therapy vectors are described in U.S. Pat. Nos. 5,354,678; 5,173,414; 5,139,941; and 5,252,479. The gene therapy vectors of the invention also include herpes vectors. Leading and preferred examples are herpes simplex virus vectors containing a sequence encoding a thymidine kinase polypeptide such as those disclosed in U.S. Pat. No. 5,288,641 and EP No. 176,170 (Roizman). Additional exemplary herpes simplex virus vectors include HFEM/ICP6-LacZ disclosed in PCT Patent No. WO 95/04139 (Wistar Institute), pHSVlac described in Geller, Science 241:1667-1669 (1988) and in PCT Patent Publication Nos. WO 90/09441 and WO 92/07945, HSV Us3::pgC-lacZ described in Fink, Human Gene Therapy 3:11-19 (1992) and HSV 7134, 2 RH 105 and GAL4 described in EP No. 453,242 (Breakefield), and those deposited with the ATCC as accession numbers ATCC VR-977 and ATCC VR-260.

Alpha virus gene therapy vectors may be employed in this invention. Preferred alpha virus vectors are Sindbis viruses vectors. Togaviruses, Semliki Forest virus (ATCC VR-67; ATCC VR-1247), Middleberg virus (ATCC VR-370), Ross River virus (ATCC VR-373; ATCC VR-1246), Venezuelan equine encephalitis virus (ATCC VR923; ATCC VR-1250; ATCC VR-1249; ATCC VR-532), and those described U.S. Pat. Nos. 5,091,309 and 5,217,879, and PCT Patent Publication No. WO 92/10578. More particularly, those alpha virus vectors described in U.S. Ser. No. 08/405,627, filed Mar. 15, 1995, and U.S. Ser. No. 08/198,450 and in PCT Patent Publication Nos. WO 94/21792, WO 92/10578, and WO 95/07994, and U.S. Pat. Nos. 5,091,309 and 5,217,879 are employable. Such alpha viruses may be obtained from depositories or collections such as the ATCC in Rockville, Md. or isolated from known sources using commonly available techniques. Preferably, alphavirus vectors with reduced cytotoxicity are used (see co-owned U.S. Ser. No. 08/679,640).

DNA vector systems such as eukaryotic layered expression systems are also useful for expressing the nucleic acids of the invention. See PCT Patent Publication No. WO 95/07994 for a detailed description of eukaryotic layered expression systems. Preferably, the eukaryotic layered expression systems of the invention are derived from alphavirus vectors and most preferably from Sindbis viral vectors. Other viral vectors suitable for use in the present invention include those derived from poliovirus, for example ATCC VR-58 and those described in Evans, Nature 339:385 (1989), and Sabin, J. Biol. Standardization 1:115 (1973); rhinovirus, for example ATCC VR-1110 and those described in Arnold, J Cell Biochem (1990) L401; pox viruses such as canary pox virus or vaccinia virus, for example ATCC VR-111 and ATCC VR-2010 and those described in Fisher-Hoch, Proc Natl Acad Sci 86 (1989) 317, Flexner, Ann NY Acad Sci 569:86 (1989), Flexner, Vaccine 8:17 (1990); in U.S. Pat. Nos. 4,603,112 and 4,769,330 and in WO 89/01973; SV40virus, for example ATCC VR-305 and those described in Mulligan, Nature 277:108 (1979) and Madzak, J Gen Vir 73:1533 (1992); influenza virus, for example ATCC VR-797 and recombinant influenza viruses made employing reverse genetics techniques as described in U.S. Pat. No. 5,166,057 and in Enami, Proc. Natl. Acad. Sci. 87:3802-3805 (1990); Enami and Palese, J. Virol. 65:2711-2713 (1991); and Luytjes, Cell 59:110 (1989), (see also McMicheal., New England J. Med. 309:13 (1983), and Yap, Nature 273:238 (1978) and Nature 277:108, 1979); human immunodeficiency virus as described in EP No. 386,882 and in Buchschacher, J. Vir. 66:2731 (1992); measles virus, for example, ATCC VR-67 and VR-1247 and those described in EP No. 440,219; Aura virus, for example, ATCC VR-368; Bebaru virus, for example, ATCC VR-600 and ATCC VR-1240; Cabassou virus, for example, ATCC VR-922; Chikungunya virus, for example, ATCC VR-64 and ATCC VR-1241; Fort Morgan Virus, for example, ATCC VR-924; Getah virus, for example, ATCC VR-369 and ATCC VR-1243; Kyzylagach virus, for example, ATCC VR-927; Mayaro virus, for example, ATCC VR-66; Mucambo virus, for example, ATCC VR-580 and ATCC VR-1244; Ndumu virus, for example, ATCC VR-371; Pixuna virus, for example, ATCC VR-372 and ATCC VR-1245; Tonate virus, for example, ATCC VR-925; Triniti virus, for example ATCC VR-469; Una virus, for example, ATCC VR-374; Whataroa virus, for example ATCC VR-926; Y-62-33 virus, for example, ATCC VR-375; O'Nyong virus, Eastern encephalitis virus, for example, ATCC VR-65 and ATCC VR-1242; Western encephalitis virus, for example, ATCC VR-70, ATCC VR-1251, ATCC VR-622 and ATCC VR-1252; and coronavirus, for example, ATCC VR-740 and those described in Hamre, Proc. Soc. Exp. Biol. Med. 121:190 (1966).

Delivery of the compositions of this invention into cells is not limited to the above mentioned viral vectors. Other delivery methods and media may be employed such as, for example, nucleic acid expression vectors, polycationic condensed DNA linked or unlinked to killed adenovirus alone, for example see U.S. Ser. No. 08/366,787, filed Dec. 30, 1994, and Curiel, Hum Gene Ther 3:147-154 (1992) ligand linked DNA, for example, see Wu, J. Biol. Chem. 264:16985-16987 (1989), eucaryotic cell delivery vehicles cells, for example see U.S. Ser. No. 08/240,030, filed May 9, 1994, and U.S. Ser. No. 08/404,796, deposition of photopolymerized hydrogel materials, hand-held gene transfer particle gun, as described in U.S. Pat. No. 5,149,655, ionizing radiation as described in U.S. Pat. No. 5,206,152 and in PCT Patent Publication No. WO 92/11033, nucleic charge neutralization or fusion with cell membranes. Additional approaches are described in Philip, Mol. Cell. Biol. 14:2411-2418 (1994) and in Woffendin, Proc. Natl. Acad. Sci. 91:1581-585 (1994). Particle mediated gene transfer may be employed, for example see U.S. provisional application No. 60/023,867. Briefly, the sequence can be inserted into conventional vectors that contain conventional control sequences for high level expression, and then be incubated with synthetic gene transfer molecules such as polymeric DNA-binding cations like polylysine, protamine, and albumin, linked to cell targeting ligands such as asialoorosomucoid, as described in Wu and Wu, J. Biol. Chem. 262:4429-4432 (1987), insulin as described in Hucked, Biochem. Pharmacol. 40:253-263 (1990), galactose as described in Plank, Bioconjugate Chem 3:533-539 (1992), lactose or transferrin. Naked DNA may also be employed. Exemplary naked DNA introduction methods are described in PCT Patent Publication No. WO 90/11092 and U.S. Pat. No. 5,580,859. Uptake efficiency may be improved using biodegradable latex beads. DNA coated latex beads are efficiently transported into cells after endocytosis initiation by the beads. The method may be improved further by treatment of the beads to increase hydrophobicity and thereby facilitate disruption of the endosome and release of the DNA into the cytoplasm. Liposomes that can act as gene delivery vehicles are described in U.S. Pat. No. 5,422,120, PCT Patent Publication Nos. WO 95/13796, WO 94/23697, and WO 91/144445, and EP No. 524,968.

As described in co-owned U.S. provisional application No. 60/023,867, on non-viral delivery, the nucleic acid sequences can be inserted into conventional vectors that contain conventional control sequences for high level expression, and then be incubated with synthetic gene transfer molecules such as polymeric DNA-binding cations like polylysine, protamine, and albumin, linked to cell targeting ligands such as asialoorosomucoid, insulin, galactose, lactose, or transferrin. Other delivery systems include the use of liposomes to encapsulate DNA comprising the gene under the control of a variety of tissue-specific or ubiquitously-active promoters. Further non-viral delivery suitable for use includes mechanical delivery systems such as the approach described in Woffendin et al., Proc. Natl. Acad. Sci. USA 91(24):11581-11585 (1994).

Moreover, the coding sequence and the product of expression of such can be delivered through deposition of photopolymerized hydrogel materials. Other conventional methods for gene delivery that can be used for delivery of the coding sequence include, for example, use of hand-held gene transfer particle gun, as described in U.S. Pat. No. 5,149,655; use of ionizing radiation for activating transferred gene, as described in U.S. Pat. No. 5,206,152 and PCT Patent Publication No. WO 92/11033. Exemplary liposome and polycationic gene delivery vehicles are those described in U.S. Pat. Nos. 5,422,120 and 4,762,915, in PCT Patent Publication Nos. WO 95/13796, WO 94/23697, and WO 91/14445, in EP No. 24,968 and in Stryer, Biochemistry, pages 236-240 (1975) W.H. Freeman, San Francisco, Szoka, Biochem. Biophys. Acta. 600:1 (1980); Bayer, Biochem. Biophys. Acta. 550:464 (1979); Rivnay, Meth. Enzymol. 149:119 (1987); Wang, Proc. Natl. Acad. Sci. 84:7851 (1987); and Plant, Anal. Biochem. 176:420 (1989).

A therapeutic agent can be administered to a patient with a measurable viral load, in a protocol that includes administration of several therapeutic agents, including an agent that reduces the viral load in the patient, an agent that stimulates CD4 T-cell production in the patient, and an agent that stimulates HIV-targeted CTLs in the patient. Any or all of these therapeutic agents can be incorporated into an appropriate pharmaceutical composition that includes a pharmaceutically acceptable carrier for the agent. Suitable carriers may be large, slowly metabolized macromolecules such as proteins, polysaccharides, polylactic acids, polyglycolic acids, polymeric amino acids, amino acid copolymers, and inactive virus particles. Such carriers are well known to those of ordinary skill in the art. Pharmaceutically acceptable salts can be used therein, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients is available in REMINGTON'S PHARMACEUTICAL SCIENCES (Mack Pub. Co., N.J. 1991). Pharmaceutically acceptable carriers in therapeutic compositions may contain liquids such as water, saline, glycerol and ethanol. Additionally, auxiliary substances, such as wetting or emulsifying agents, pH buffering substances, and the like, may be present in such vehicles. Typically, the therapeutic compositions are prepared as injectables, either as liquid solutions or suspensions; solid forms suitable for solution in, or suspension in, liquid vehicles prior to injection may also be prepared. Liposomes are included within the definition of a pharmaceutically acceptable carrier. The term “liposomes” refers to, for example, the liposome compositions described in U.S. Pat. No. 5,422,120, WO 95/13796, WO 94/23697, WO 91/14445 and EP 524,968 B1. Liposomes may be pharmaceutical carriers for the small molecules, polypeptides or polynucleotides of the invention, or for combination of these therapeutics.

Additionally, administration of the therapeutic agents of the invention can be accomplished for example, in a simultaneous administration, in sequential administration, and with the same or different pharmaceutically acceptable carriers, as is appropriate for best accomplishing the goal of reducing the viral load of the patient. Thus, for example, the viral load reducer may be administered first, followed by either a simultaneous or sequential administration of a CD4 T-cell inducer and a CTL inducer. It is also envisioned that a repeat administration of a viral load reducer might be necessary, in addition to repeated administration of the agent capable of increasing the patients CD4 T-cell count and CTLs.

Further, a therapeutic composition can be administered that includes all the therapeutic agents necessary to achieve the therapeutic goals of the therapy. Thus, the therapeutic composition could include a viral load reducer, an agent to induce CD4 T-cells, and an agent to induce CTLs. For example, a protease inhibitor in combination with a reverse transcriptase inhibitor, both chemotherapeutic agents could be administered with a naked DNA encoding IL-2 for expression in the patient, also in combination with a DNA vaccine that includes a polynucleotide encoding the HIV p24 subunit, also for expression in the patient.

Any therapeutic of the invention, including, for example, polynucleotides for expression in the patient, or ribozymes or antisense oligonucleotides, can be formulated into an enteric coated tablet or gel capsule according to known methods in the art. These are described in the following patents: U.S. Pat. No. 4,853,230, EP 225,189, AU 9,224,296, AU 9,230,801, and WO 92/14452. Such a capsule is administered orally to be targeted to the jejunum. At 1 to 4 days following oral administration expression of the polypeptide, or inhibition of expression by, for example a ribozyme or an antisense oligonucleotide, is measured in the plasma and blood, for example by antibodies to the expressed or non-expressed proteins.

Administration of a therapeutic of the invention, includes administering a therapeutically effective dose of the therapeutic, by a means considered or empirically determined to be effective for inducing the desired, therapeutic effect in the patient. Both the dose and the administration means can be determined based on the specific qualities of the therapeutic, the condition of the patient, the progression of the disease, and other relevant factors. Administration for the therapeutic agents of the invention can include, for example, local or systemic administration, including for example parenteral administration, including injection, topical administration, oral administration, catheterization, laser-created perfusion channels, a particle gun, and a pump. Parenteral administration can be, for example, intravenous, subcutaneous, intradermal, or intramuscular, administration.

Diagnosis of the HIV infection can be made using an antibody specific for the HIV, but diagnosis can be achieved at an earlier stage of the disease using nucleic acid hybridization techniques, including, for example, use of nucleic acid probes, for example, as described in EP 617, 132, PCR, as described in WO 94/20640, for example, and bDNA technology. The most sensitive of these techniques is bDNA technology, as described in as described in WO 92/02526 and U.S. Pat. Nos. 5,451,503 and 4,775,619. Diagnosis can include measuring a viral load of a patient, for example measuring an amount of HIV RNA in plasma, cells or tissue from a patient. Subsequent monitoring of the patient can include periodic diagnostic tests following administration of the vaccination therapy.

The therapeutics of the invention can be administered in a therapeutically effective dosage and amount, in the process of a therapeutically effective protocol for treatment of the patient. The initial and any subsequent dosages administered will depend upon the patient's age, weight, condition, and the disease, disorder or biological condition being treated. Depending on the therapeutic, the dosage and protocol for administration will vary, and the dosage will also depend on the method of administration selected, for example, local or systemic administration.

For polypeptide therapeutics, for example, IL-2, or other cytokine, the dosage can be in the range of about 5 μg to about 500 μg/kg of patient body weight, also about 50 μg to about 5 mg/kg, also about 100 μg to about 500 μg/kg of patient body weight, and about 200 to about 250 ug/kg.

For polynucleotide therapeutics, depending on the expression of the polynucleotide in the patient, for tissue targeted administration, vectors containing expressable constructs of coding sequences, or non-coding sequences can be administered in a range of about 100 ng to about 200 mg of DNA for local administration in a gene therapy protocol, also about 500 ng to about 50 mg, also about 1 ug to about 2 mg of DNA, about 5 ug of DNA to about 500 ug of DNA, and about 20 ug to about 100 ug during a local administration in a gene therapy protocol, and for example, a dosage of about 500 ug, per injection or administration.

Non-coding sequences that act by a catalytic mechanism, for example, catalytically active ribozymes may require lower doses than non-coding sequences that are held to the restrictions of stoichometry, as in the case of, for example, antisense molecules, although expression limitations of the ribozymes may again raise the dosage requirements of ribozymes being expressed in vivo in order that they achieve efficacy in the patient. Factors such as method of action and efficacy of transformation and expression are therefore considerations that will effect the dosage required for ultimate efficacy for DNA and nucleic acids. Where greater expression is desired, over a larger area of tissue, larger amounts of DNA or the same amounts readministered in a successive protocol of administrations, or several administrations to different adjacent or close tissue portions of for example, a tumor site, may be required to effect a positive therapeutic outcome.

For administration of small molecule therapeutics, depending on the potency of the small molecule, the dosage may vary. For a very potent inhibitor, microgram (μ) amounts per kilogram of patient may be sufficient, for example, in the range of about 1 μg/kg to about 500 mg/kg of patient weight, and about 100 μg/kg to about 5 mg/kg, and about 1 μg/kg to about 50 μg/kg, and, for example, about 10 ug/kg. For administration of peptides and peptoids the potency also affects the dosage, and may be in the range of about 1 μg/kg to about 500 mg/kg of patient weight, and about 100 μg/kg to about 5 mg/kg, and about 1 μg/kg to about 50 μg/kg, and a usual dose might be about 10 ug/kg.

In all cases, routine experimentation in clinical trials will determine specific ranges for optimal therapeutic effect, for each therapeutic, each administrative protocol, and administration to specific patients will also be adjusted to within effective and safe ranges depending on the patient condition and responsiveness to initial administrations.

Further objects, features, and advantages of the present invention will become apparent from the detailed description. It should be understood, however, that the detailed description, while indicating preferred embodiments of the invention, is given by way of illustration only, since various changes and modifications within the spirit and scope of the invention will become apparent to those skilled in the art from this detailed description. Also, the invention is not limited by any theories of mechanism of the method of the invention.

The invention described herein draws on previously published work and pending patent applications. By way of example, such work consists of scientific papers, patents or pending patent applications. All such published work cited herein are hereby incorporated by reference in its entirety.

The present invention will now be illustrated by reference to the following examples which set forth particularly advantageous embodiments. However, it should be noted that these embodiments are illustrative and are not to be construed as restricting the invention in any way.

### EXAMPLE 1

A patient is diagnosed with a viral load of about 20,000 copies of RNA per mL of plasma. The patient is administered a combination of zidovudine with lamivudine and Indivinavir, and also intravenous injections of an organic small molecule inhibitor of a tat/tar interaction, and the viral load in the patient is reduced to an undectable level.

The patient is then administered a polynucleotide encoding IL-2 des Ala-Ser 125 in a formulation for gene delivery to cells by an inhalation therapy protocol for about a week, by use of an aerosol spray formulation administered hourly during the waking hours of the day.

During the last few days of the week of IL-2 administration, the patient is vaccinated with a DNA vaccine made up of a polynucleotide encoding the p24 subunit of HIV. The IL-2 gene therapy is repeated, followed by another vaccination with p24 subunit DNA. The patient is monitored for viral load, and CD4 T-cells, and the treatment is repeated until the viral load remains undectable for an extended period of time, and CD4 T-cell count has returned to normal or near normal levels.

What is claimed:

1. A method of eliminating human immunodeficiency virus (HIV) in an HIV-infected patient, the patient having a measurable viral load, comprising the steps:
(a) reducing the viral load in the patient by administration of a first therapeutic agent,
(b) administering a second therapeutic agent capable of increasing a count of a T-cell lymphocyte expressing a cluster of differentiation-4 antigen (CD4 T-cell) in the patient, and
(c) administering a third therapeutic agent capable of increasing a number of cytotoxic T-cell lymphocytes (CTLs) in the patient.

2. A combination therapeutic agent for eliminating HIV in an HIV-infected patient having a measurable viral load comprising a viral load reducer, a CD4 T-cell inducer, and a vaccine capable of increasing a CTL count in the patient.
